SEQUENCE LISTING (67 sequences)

SEQ ID NO: 1 - DNA, 16 bp, Artificial Sequence
  Exemplary Immunostimulatory Oligonucleotide human c-myc
taacgttgaggggcat

SEQ ID NO: 2 - DNA, 20 bp, Artificial Sequence
  Synthetic oligodeoxynucleotide
ttccatgacgttcctgacgt

SEQ ID NO: 3 - DNA, 19 bp, Artificial Sequence
  Synthetic oligodeoxynucleotide
tccatgacgttcctgacgt

SEQ ID NO: 4 - DNA, 16 bp, Artificial Sequence
  Exemplary Immunostimulatory Oligonucleotide
taacgttgaggggcat

SEQ ID NO: 5 - DNA, 20 bp, Artificial Sequence
  Exemplary Immunostimulatory Oligonucleotide
tccatgacgttcctgacgtt

SEQ ID NO: 6 - DNA, 16 bp, Artificial Sequence
  Exemplary Immunostimulatory Oligonucleotide
taagcatacggggtgt

SEQ ID NO: 7 - DNA, 24 bp, Artificial Sequence
  Exemplary Immunostimulatory Oligonucleotide
gatgctgtgtcggggtctccgggc

SEQ ID NO: 8 - DNA, 24 bp, Artificial Sequence
  Exemplary Immunostimulatory Oligonucleotide
tcgtcgttttgtcgttttgtcgtt

SEQ ID NO: 9 - DNA, 24 bp, Artificial Sequence
  Exemplary Immunostimulatory Oligonucleotide
tcgtcgttttgtcgttttgtcgtt

SEQ ID NO: 10 - DNA, 20 bp, Artificial Sequence
  Exemplary Immunostimulatory Oligonucleotide
tccaggacttctctcaggtt

SEQ ID NO: 11 - DNA, 18 bp, Artificial Sequence
  Exemplary Immunostimulatory Oligonucleotide
tctcccagcgtgcgccat

SEQ ID NO: 12 - DNA, 20 bp, Artificial Sequence
  Exemplary Immunostimulatory Oligonucleotide murine Intracellular Adhesion Molecule-1
tgcatcccccaggccaccat

SEQ ID NO: 13 - DNA, 20 bp, Artificial Sequence
  Exemplary Immunostimulatory Oligonucleotide human Intracellular Adhesion Molecule-1
gcccaagctggcatccgtca

SEQ ID NO: 14 - DNA, 15 bp, Artificial Sequence
  Exemplary Immunostimulatory Oligonucleotide human erb-B-2
ggtgctcactgcggc

SEQ ID NO: 15 - DNA, 16 bp, Artificial Sequence
  Exemplary Immunostimulatory Oligonucleotide human c-myc
aaccgttgaggggcat

SEQ ID NO: 16 - DNA, 24 bp, Artificial Sequence
  Exemplary Immunostimulatory Oligonucleotide human c-myc
tatgctgtgccggggtcttcgggc

SEQ ID NO: 17 - DNA, 18 bp, Artificial Sequence
  Exemplary Immunostimulatory Oligonucleotide
gtgccggggtcttcgggc

SEQ ID NO: 18 - DNA, 18 bp, Artificial Sequence
  Exemplary Immunostimulatory Oligonucleotide human Insulin Growth Factor 1-Receptor
ggaccctcctccggagcc

SEQ ID NO: 19 - DNA, 18 bp, Artificial Sequence
  Exemplary Immunostimulatory Oligonucleotide human Insulin Growth Factor 1-Receptor
tcctccggagccagactt

SEQ ID NO: 20 - DNA, 15 bp, Artificial Sequence
  Exemplary Immunostimulatory Oligonucleotide human Epidermal Growth Factor-Receptor
aacgttgaggggcat

SEQ ID NO: 21 - DNA, 15 bp, Artificial Sequence
  Exemplary Immunostimulatory Oligonucleotide human Epidermal Growth Factor-Receptor
ccgtggtcatgctcc

SEQ ID NO: 22 - DNA, 21 bp, Artificial Sequence
  Exemplary Immunostimulatory Oligonucleotide human Vascular Endothelial Growth Factor
cagcctggctcaccgccttgg

SEQ ID NO: 23 - DNA, 20 bp, Artificial Sequence
  Exemplary Immunostimulatory Oligonucleotide murine Phosphokinase C-alpha
cagccatggttccccccaac

SEQ ID NO: 24 - DNA, 20 bp, Artificial Sequence
  Exemplary Immunostimulatory Oligonucleotide
gttctcgctggtgagtttca

SEQ ID NO: 25 - DNA, 18 bp, Artificial Sequence
  Exemplary Immunostimulatory Oligonucleotide human Bcl-2
tctcccagcgtgcgccat

SEQ ID NO: 26 - DNA, 15 bp, Artificial Sequence
  Exemplary Immunostimulatory Oligonucleotide human C-Raf-s
gtgctccattgatgc

SEQ ID NO: 27 - DNA, 33 bp, Artificial Sequence
  Exemplary Immunostimulatory Oligonucleotide human Vascular Endothelial Growth Factor Receptor-1
gaguucugaugaggccgaaaggccgaaagucug

SEQ ID NO: 28 - DNA, 15 bp, Artificial Sequence
  Exemplary Immunostimulatory Oligonucleotide
aacgttgaggggcat

SEQ ID NO: 29 - DNA, 16 bp, Artificial Sequence
  Exemplary Immunostimulatory Oligonucleotide
caacgttatggggaga

SEQ ID NO: 30 - DNA, 21 bp, Artificial Sequence
  Synthetic oligonucleotide
ggaaucuuauauuugauccaa

SEQ ID NO: 31 - DNA, 23 bp, Artificial Sequence
  Synthetic oligonucleotide
uuggaucaaauauaagauucccu

SEQ ID NO: 32 - DNA, 21 bp, Artificial Sequence
  Synthetic oligonucleotide
ggaaucuuauauuugauccaa

SEQ ID NO: 33 - DNA, 21 bp, Artificial Sequence
  Synthetic oligonucleotide
ggaaucuuauauuugauccaa

SEQ ID NO: 34 - DNA, 21 bp, Artificial Sequence
  Synthetic oligonucleotide
ggaaucuuauauuugauccaa

SEQ ID NO: 35 - DNA, 21 bp, Artificial Sequence
  Synthetic oligonucleotide
ggaaucuuauauuugauccaa

SEQ ID NO: 36 - DNA, 21 bp, Artificial Sequence
  Synthetic oligonucleotide
ggaaucuuauauuugauccaa

SEQ ID NO: 37 - DNA, 21 bp, Artificial Sequence
  Synthetic oligonucleotide
ggaaucuuauauuugauccaa

SEQ ID NO: 38 - DNA, 21 bp, Artificial Sequence
  Synthetic oligonucleotide
ggaaucuuauauuugauccaa

SEQ ID NO: 39 - DNA, 21 bp, Artificial Sequence
  Synthetic oligonucleotide
guauugucccuauaggcuutt

SEQ ID NO: 40 - DNA, 21 bp, Artificial Sequence
  Synthetic oligonucleotide
aagccuauagggacaauactt

SEQ ID NO: 41 - DNA, 21 bp, Artificial Sequence
  Synthetic oligonucleotide
guauugucccuauaggcuutt

SEQ ID NO: 42 - DNA, 21 bp, Artificial Sequence
  Synthetic oligonucleotide
cuuacgcugaguacuucgatt

SEQ ID NO: 43 - DNA, 21 bp, Artificial Sequence
  Synthetic oligonucleotide
ucgaaguacucagcguaagtt

SEQ ID NO: 44 - DNA, 24 bp, Artificial Sequence
  Synthetic oligonucleotide
ucgaaguacucagcguaagdtsdt

SEQ ID NO: 45 - DNA, 21 bp, Artificial Sequence
  Synthetic oligonucleotide
cuuacgcugaguacuucgatt

SEQ ID NO: 46 - DNA, 21 bp, Artificial Sequence
  Synthetic oligonucleotide
aagccuauagggacaauactt

SEQ ID NO: 47 - DNA, 21 bp, Artificial Sequence
  Synthetic oligonucleotide
acgaaugaguuuugugcuutt

SEQ ID NO: 48 - DNA, 21 bp, Artificial Sequence
  Synthetic oligonucleotide
aagcacaaaacucauucgutt

SEQ ID NO: 49 - DNA, 21 bp, Artificial Sequence
  Synthetic oligonucleotide
gaagcagcuucaggaugagtt

SEQ ID NO: 50 - DNA, 21 bp, Artificial Sequence
  Synthetic oligonucleotide
cucauccugaagcugcuuctt

SEQ ID NO: 51 - DNA, 21 bp, Artificial Sequence
  Synthetic oligonucleotide
ccugcauugucaacuaucatt

SEQ ID NO: 52 - DNA, 21 bp, Artificial Sequence
  Synthetic oligonucleotide
ugauaguugacaaugcaggtt

SEQ ID NO: 53 - DNA, 21 bp, Artificial Sequence
  Synthetic oligonucleotide
acgaaugaguuuugugcuutt

SEQ ID NO: 54 - DNA, 21 bp, Artificial Sequence
  Synthetic oligonucleotide
acgaaugaguuuugugcuutt

SEQ ID NO: 55 - DNA, 21 bp, Artificial Sequence
  Synthetic oligonucleotide
acgaaugaguuuugugcuutt

SEQ ID NO: 56 - DNA, 21 bp, Artificial Sequence
  Synthetic oligonucleotide
acgaaugaguuuugugcuutt

SEQ ID NO: 57 - DNA, 21 bp, Artificial Sequence
  Synthetic oligonucleotide
acgaaugaguuuugugcuutt

SEQ ID NO: 58 - DNA, 21 bp, Artificial Sequence
  Synthetic oligonucleotide
acgaaugaguuuugugcuutt

SEQ ID NO: 59 - DNA, 21 bp, Artificial Sequence
  Synthetic oligonucleotide
acgaaugaguuuugugcuutt

SEQ ID NO: 60 - DNA, 21 bp, Artificial Sequence
  Synthetic oligonucleotide
acgaaugaguuuugugcuutt

SEQ ID NO: 61 - DNA, 21 bp, Artificial Sequence
  Synthetic oligonucleotide
acgaaugaguuuugugcuutt

SEQ ID NO: 62 - DNA, 21 bp, Artificial Sequence
  Synthetic oligonucleotide
acgaaugaguuuugugcuutt

SEQ ID NO: 63 - DNA, 21 bp, Artificial Sequence
  Synthetic oligonucleotide
acgaaugaguuuugugcuutt

SEQ ID NO: 64 - DNA, 21 bp, Artificial Sequence
  Synthetic oligonucleotide
acgaaugaguuuugugcuutt

SEQ ID NO: 65 - DNA, 21 bp, Artificial Sequence
  Synthetic oligonucleotide
acgaaugaguuuugugcuutt

SEQ ID NO: 66 - DNA, 21 bp, Artificial Sequence
  Synthetic oligonucleotide
acgaaugaguuuugugcuutt

SEQ ID NO: 67 - DNA, 21 bp, Artificial Sequence
  Synthetic oligonucleotide
aagcacaaaacucauucgutt

(19) United States
(12) Patent Application Publication
(10) Pub. No.: US 2010/0222417 A1
(43) Pub. Date: Sep. 2, 2010

(54) Compostions and methods for enhancing oligonucleotide delivery across and into epithelial tissues

(75) Inventors: Tracy Zimmermann, Cambridge, MA (US); Kevin Fitzgerald, Cambridge, MA (US); Muthiah Manoharan, Cambridge, MA (US)

Correspondence Address: Nixon Peabody LLP, 401 9th Street N.W., Suite 900, Washington, DC, 20004, US

(73) Assignee: Alnylam Pharmaceuticals, Cambridge, MA (US)

(21) Appl. No.: 12/591,629

(22) Filed: Nov. 25, 2009

Related U.S. Application Data
(60) Provisional application No. 61/200,256, filed on Nov. 26, 2008.
(60) Provisional application No. 61/143,634, filed on Jan. 9, 2009.

(51) Int. Cl. A61K 31/7088; A61K 31/56

(52) U.S. Cl. 514/44 R; 514/182

(57) ABSTRACT

The present invention relates to the delivery of oligonucleotide across and into epithelial tissues by conjugating the oligonucleotide with a lipophile and/or coadministering with a penetration enhancer. In particular, the present invention provides a composition comprising a conjugated siRNA, which are advantageous for the in vivo delivery of nucleic acids across and into epithelial tissue. Additionally, the present invention provides methods of improving delivery of oligonucleotides across the epithelial tissues with the aid of a mechanical enhancer.

### PRIORITY CLAIM

This application claims benefit under 35 U.S.C. §119(e) to U.S. Provisional Application No. 61/200,256, filed Nov. 26, 2008 and U.S. Provisional Application No. 61/143,634, filed Jan. 9, 2009, both of which are herein incorporated by reference in their entirety.

### TECHNICAL FIELD

The present invention relates to the delivery of oligonucleotide across and into epithelial tissues by conjugating the oligonucleotide with a lipophile and optionally coadministering with a penetration enhancer or a mechanical enhancer. In particular, the present invention provides a composition comprising a conjugated siRNA optionally coadministered with a penetration enhancer, which is advantageous for the in vivo delivery of nucleic acids across and into epithelial tissue. Additionally, the present invention provides methods of improving delivery of oligonucleotides across the epithelial tissues with the aid of a mechanical enhancer.

### BACKGROUND

Recently, double-stranded RNA molecules (dsRNA) have been shown to block gene expression in a highly conserved regulatory mechanism known as RNA interference (RNAi). WO 99/32619 (Fire et al.) discloses the use of a dsRNA of at least 25 nucleotides in length to inhibit the expression of genes in C. elegans. dsRNA has also been shown to degrade target RNA in other organisms, including plants (see, e.g., WO 99/53050, Waterhouse et al.; and WO 99/61631, Heifetz et al.), Drosophila (see, e.g., Yang, D., et al., Curr. Biol. (2000) 10:1191-1200), and mammals (see WO 00/44895, Limmer; and DE 101 00 586.5, Kreutzer et al.). This natural mechanism has now become the focus for the development of a new class of pharmaceutical agents for treating disorders that are caused by the aberrant or unwanted regulation of a gene.

Despite significant advances in the field of RNAi and advances in the treatment of pathological processes, there remains a need for formulations that can selectively and efficiently deliver agents to cells where silencing can then occur.

While delivery of oligonucleotides across plasma membranes in vivo has been achieved using vector-based delivery systems, high-pressure intravenous injections of oligonucleotides and various chemically-modified oligonucleotides, including cholesterol-conjugated, lipid encapsulated and antibody-mediated oligonucleotides, to date, delivery remains the largest obstacle for in vivo oligonucleotide therapeutics. Specifically, delivery of oligonucleotides across the epithelial tissues has not been extensively studied.

### BRIEF SUMMARY

The present invention is based on the discovery that lipophilic conjugated oligonucleotides can be delivered across and into the epithelial tissues. The delivery is further enhanced with the use of said oliogonucleotides with a penetration enhancer. The inventors also found that cleavable linkers can improve the delivery across and into the epithelial tissues.

In one embodiment, the invention relates to a method for enhancing delivery of an oligonucleotide into and across one or more layers of an animal epithelial tissue, the method comprising administering to the epithelial tissues a lipophilic conjugated oligonucleotide.

In one embodiment, the invention relates to a compostion for delivery of oligonucleotides across and into the epithelial tissues comprising a lipophilic conjugated oligonucleotide, a penetration enhancer, and a mechanical enhancer.

In one embodiment, the invention relates to a method for treating papillomavirus (HPV).

### BRIEF DESCRIPTION OF THE DRAWINGS

U.S. Provisional Application No. 61/200,256, incorporated herein by reference and claimed as a priority document to this application, contains FIGS. 1-8, executed in color. Applicants do not believe that color figures are necessary to understand the invention and the concepts illustrated in the figures; however, if needed, the color figures may be referenced in the priority document.

FIG. 1 illustrates the distribution of siRNAs with lipophilic conjugates without ethanol: (a) S/5′ Cy3-AS-ApoB (AD-18560), (b) 3′Cholesterol-S/5′Cy3-AS-ApoB (AD-18117), (c) 3′Lithocholicoleoyl-S/5′Cy3-AS-ApoB (AD-18565), (d) 3′Disulfide-Cholesterol-S/5′Cy3-AS-ApoB (AD-18563), (e) 3′C16-Cholesterol-S/5′Cy3-AS-ApoB (AD-18561), (f) 3′PEG4-Cholesterol-S/5′Cy3-AS-ApoB (AD-18562), (g) 3′C22-S/5′Cy3-AS-ApoB (AD-18564), (h) 3′Cholanic-S/5′Cy3-AS-ApoB (AD-18566).

FIG. 2 depicts the distribution K14 siRNA with and without cholesterol for two animals: (a) and (c) S/5′Cy3-AS K14, (b) and (d) 3′Cholesterol-S/5′Cy3-AS K14

FIG. 3 shows a comparison between conjugate vs. non-conjugate: (a) S/5′Cy3-AS Luc+2.5% EtOH, 4 hours, (b) 3′Cholesterol-S/5′Cy3-AS Luc+2.5% EtOH, 4 hours.

FIG. 4 shows a comparison between conjugate vs. non-conjugate: (a) S/5′Cy3-AS Luc+2.5% EtOH, 4 hours, (b) 3′Cholesterol-S/5′Cy3-AS Luc+2.5% EtOH 4.5 hours, (c) S/5′Alexa488-AS Luc+2.5% EtOH, 4 hours, (d) 3′Cholesterol-S/5′Alexa488-AS Luc+2.5% EtOH, 4.5 hours.

FIG. 5 compares the different concentrations of EtOH at 4 hours: (a) 3′Cholesterol-S/5′Alexa488-AS Luc+0.5% EtOH, (b) 3′Cholesterol-S/5′Alexa488-AS Luc+1% EtOH, (c) 3′Cholesterol-S/5′Alexa488-AS+2.5% EtOH.

FIG. 6 compares the different concentrations of EtOH at 18 hours: (a) 3′Cholesterol-S/5′Alexa488-AS Luc+0.5% EtOH, (b) 3′Cholesterol-S/5′Alexa488-AS Luc+1% EtOH, (c) 3′Cholesterol-S/5′Alexa488-AS Luc+2.5% EtOH.

FIG. 7 compares the different concentrations of EtOH at 18 hours in the basal epithelia layer: (a) 3′Cholesterol-S/5′Alexa488-AS Luc+0.5% EtOH, (b) 3′Cholesterol-S/5′Alexa488-AS Luc+1% EtOH, (c) 3′Cholesterol-S/5′Alexa488-AS Luc+2.5% EtOH.

FIG. 8 shows the permeation of siRNA at 22% and 10% Ethanol at 17.5 hours: (a) Acetyl-cysteine/2×PBS/Chol-Alexa Luc+22% EtOH, (b) Acetyl-cysteine/2×PBS/Chol-Alexa Luc+10% EtOH.

FIG. 9 is a bar graph showing the efficacy in vivo of Chol-K14 siRNA relative to Chol-E6AP siRNA and PBS in the vaginal canal.

FIG. 10 contains bar graphs showing the efficacy in vivo of Chol-K14 relative to Chol-E6AP and PBS with and without EtOH: (a) Relative K14 mRNA in vaginal canal (VC) for Chol-conjugate w/Cytobrush®, No EtOH, (b) Relative K14 mRNA in vaginal canal (VC) for Chol-conjugate with Cytobrush®, +5% EtOH:

FIG. 11 contains bar graphs showing the efficacy in vivo of Chol-K14 relative to Chol-E6AP and PBS at 24 and 48 hour: (a) Relative K14 mRNA in VC for Chol-conjugate w/Cytobrush®, +5% EtOH, at 24 hours, (b) Relative K14 mRNA in VC for Chol-conjugate w/Cytobrush®, +5% EtOH, at 48 hours.

FIG. 12 contains bar graphs showing the efficacy in vivo of Chol-K14 relative to Chol-E6AP and PBS in two different mouse models: (a) Relative K14 mRNA in VC for Chol-conjugate w/Cytobrush®, +5% EtOH, Balb/C, (b) Relative K14 mRNA in VC for Chol-conjugate w/Cytobrush®, +5% EtOH, C57BL/6.

FIG. 13 contains bar graphs showing the efficacy in vivo of Chol-K14 relative to Chol-Lamin A/C: (a) Normalized K14 in VC for Chol-conjugate+5% EtOH with Cytobrush® as % Lamin ACcontrol, (b) Normalized K14 in VC for Chol-conjugate+5% EtOH with no Cytobrush® as % Lamin ACcontrol.

FIG. 14 is a bar graph illustrating no non-specific K14 KD with E6AP-Chol or Nectin-Chol siRNAs, Balb/C with Cytobrush® Chol-siRNA+5% EtOH in the vaginal canal.

FIG. 15 is a bar graph illustrating free uptake of various lipophilic conjugated siRNAs in 1° Human Keratinocytes.

### DETAILED DESCRIPTION

The present invention is based on the discovery that lipophilic conjugated oligonucleotides can be delivered across and into the epithelial tissues. The delivery is further enhanced with the use of said oliogonucleotides with a penetration enhancer. The inventors also found that certain linkers can improve the delivery across and into the epithelial tissues.

In one embodiment, the invention relates to a method for enhancing delivery of an oligonucleotide into and across one or more layers of an animal epithelial tissue, the method comprising administering to the epithelial tissue a lipophilic conjugated oligonucleotide.

The term “epithelial tissue”, as used herein, refers to tissue on the exterior of the body of a subject, or layering its interior surfaces, that is covered by continuous cellular sheets known as epithelial membranes (or epithelia) and the various glands (both exocrine and endocrine) that develop there from, and includes, without limitation, any or all of the following: endothelium, mesothelium, and skin (including epidermis and dermis). The cellular (typically avascular) layer covering all the free surfaces, cutaneous, mucous, and serous, including the glands and other structures derived there from. Epithelial tissue present squamous, cuboidal, and/or columnar cells upon histological examination. In addition, epithelial tissue may be described as simple, stratified or pseudostratified. Examples of cells of the epidermis include, without limitation, Langerhans cells, keratinocytes, and melanocytes. Keratinocytes are committed cells, arising deep in the epidermis, that undergo gradual transformation into scales of keratin as they become displaced toward the surface. In one embodiment, the epithelial tissue is in the vaginal canal, urinary tract, lung, buccal mucosa, and skin.

The lipophilic conjugate of the invention includes, but not limited to steroid (i.e. cholesterol, lithocholic acid and stigmasterol), aliphatic chain, phospholipid, polyethylene glycol (i.e. PEG, mw 100-40K or methylPEG, mw 120-40K), lipophilic vitamins (i.e. vitamin E or vitamin E homologs such as alpha-tocopherol, beta-tocopherol, gamma-tocopherol, delta-tocopherol, alpha-tocotrienol, beta-tocotrienol, gamma-tocotrienol, and delta-tocotrienol), coenzyme Q10, carotenoids, alpha-lipoic acid, and essential fatty acids. The lipophilic moiety can be chosen, for example, from the group consisting of a lipid, cholesterol, oleyl, retinyl, cholesteryl residues, cholic acid, adamantane acetic acid, 1-pyrene butyric acid, dihydrotestosterone, 1,3-Bis-O (hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine. The preferred lipophilic conjugates are cholesterol, C-14 to C-22 aliphatic chain, and lithocolic acid.

The delivery to the epithelial tissues is further enhanced with the use of cleavable linker such as a redox cleavable linking group (i.e. disulfide), an acid cleavable linking group (i.e. hydrazone, ester, anhydride), an esterase cleavable linking group (i.e. an ester group), a phosphatase cleavable linking group (a phosphate group), or a peptidase cleavable linking group (a peptide bond).

In one embodiment, cleavable linking groups can be a reductively cleavable linking group selected from the group consisting of:

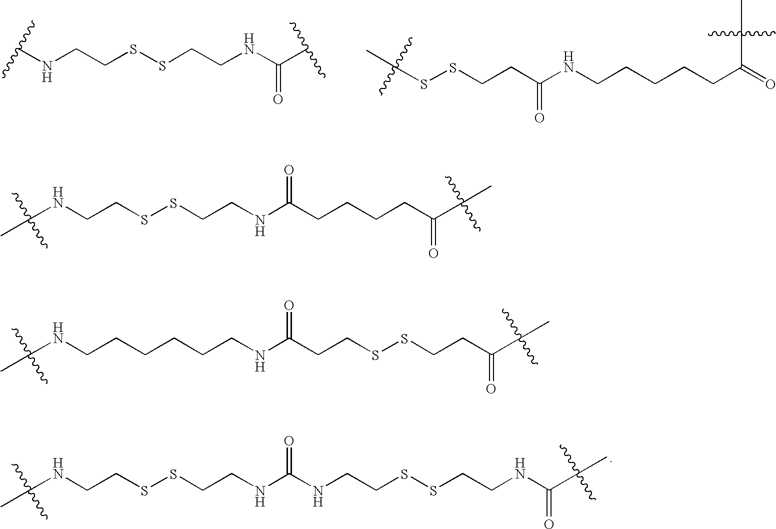

In one embodiment, the linker-conjugate of the invention is selected from the group consisting of:

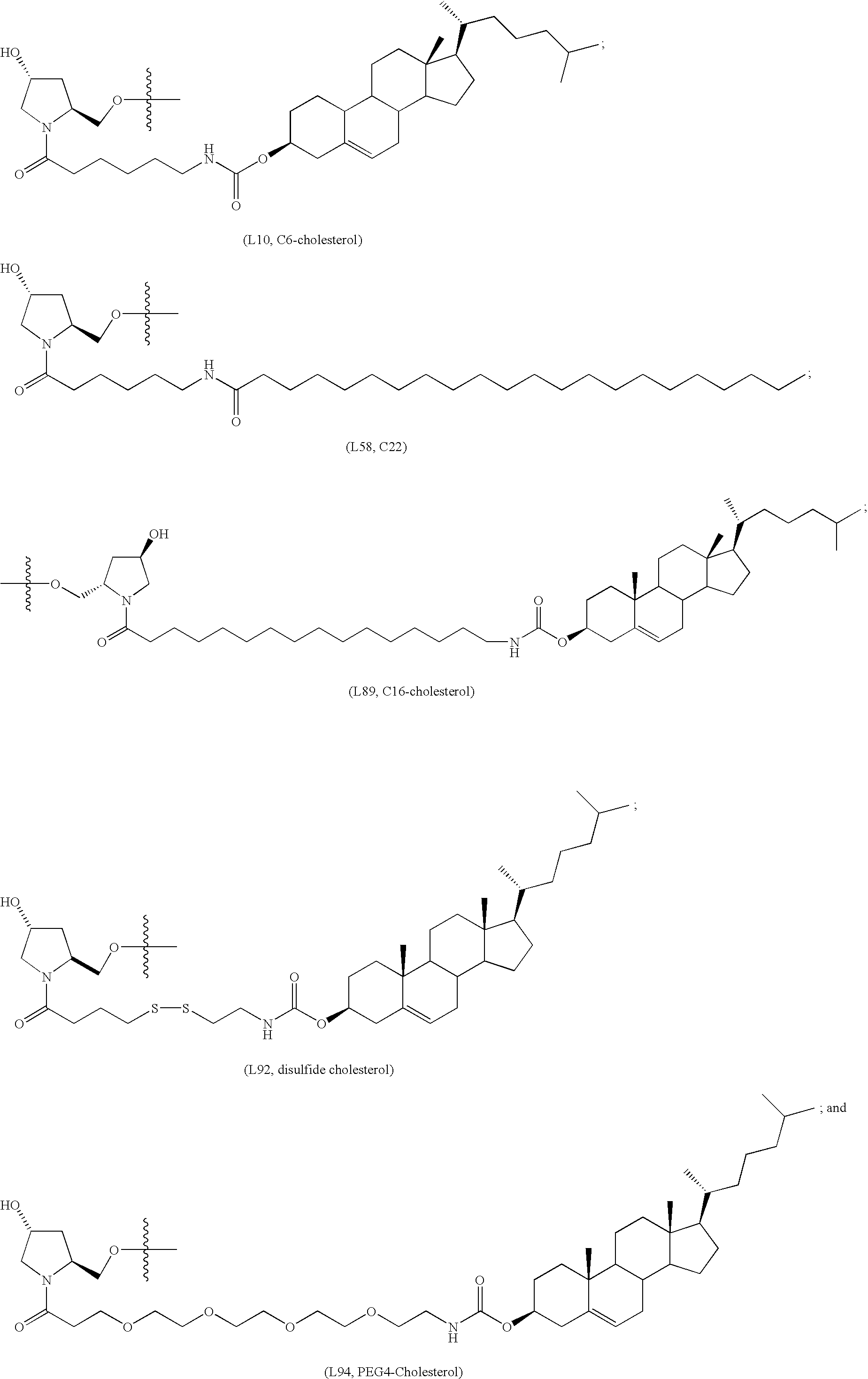

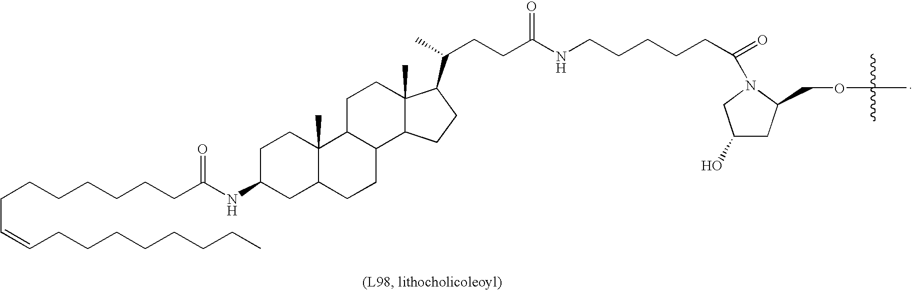

In one embodiment, the conjugated oligonucleotide is coadmistered with a penetration enhancer. Various types of penetration enhancers may be used to enhance transdermal transport of drugs. Penetration enhancers can be divided into chemical enhancers and mechanical enhancers, each of which is described in more detail below.

Chemical enhancers enhance molecular transport rates across tissues or membranes by a variety of mechanisms. In the present invention, chemical enhancers are preferably used to decrease the barrier properties of the stratum corneum. Drug interactions include modifying the drug into a more permeable state (a prodrug), which would then be metabolized inside the body back to its original form (6-fluorouracil, hydrocortisone) (Hadgraft, 1985); or increasing drug solubilities (ethanol, propylene glycol). Despite a great deal of research (well over 200 compounds have been studied) (Chattaraj and Walker, 1995), there are still no universally applicable mechanistic theories for the chemical enhancement of molecular transport. Most of the published work in chemical enhancers has been done largely based on experience and on a trial-and-error basis (Johnson, 1996).

In one embodiment, chemical enhancers can include cationic, anionic, and nonionic surfactants (sodium dodecyl sulfate, polyoxamers); fatty acids and alcohols (ethanol, oleic acid, lauric acid, liposomes); anticholinergic agents (benzilonium bromide, oxyphenonium bromide); alkanones (n-heptane); amides (urea, N,N-diethyl-m-toluamide); fatty acid esters (n-butyrate); organic acids (citric acid); polyols (ethylene glycol, glycerol); sulfoxides (dimethylsulfoxide); and terpenes (cyclohexene) (Hadgraft and Guy, 1989; Walters, 1989; Williams and Barry, 1992; Chattaraj and Walker, 1995). Most of these enhancers interact either with the skin or with the drug. Those enhancers interacting with the skin are herein termed “lipid permeation enhancers”, and include interactions with the skin include enhancer partitioning into the stratum corneum, causing disruption of the lipid bilayers (azone, ethanol, lauric acid), binding and disruption of the proteins within the stratum corneum (sodium dodecyl sulfate, dimethyl sulfoxide), or hydration of the lipid bilayers (urea, benzilonium bromide). Other chemical enhancers work to increase the transdermal delivery of a drug by increasing the drug solubility in its vehicle (hereinafter termed “solubility enhancers”). Lipid permeation enhancers, solubility enhancers, and combinations of enhancers (also termed “binary systems”) are discussed in more detail below.

Chemicals which enhance permeability through lipids are known and commercially available. For example, ethanol increases the solubility of drugs up to 10.000-fold and yield a 140-fold flux increase of estradiol, while unsaturated fatty acids increase the fluidity of lipid bilayers (Bronaugh and Maibach, editors (Marcel Dekker 1989) pp. 1-12. Examples of fatty acids which disrupt lipid bilayer include linoleic acid, capric acid, lauric acid, and neodecanoic acid, which can be in a solvent such as ethanol or propylene glycol. Evaluation of published permeation data utilizing lipid bilayer disrupting agents agrees very well with the observation of a size dependence of permeation enhancement for lipophilic compounds. The permeation enhancement of three bilayer disrupting compounds, capric acid, lauric acid, and neodecanoic acid, in propylene glycol has been reported by Aungst, et al. Pharm. Res. 7, 712-718 (1990). They examined the permeability of four lipophilic compounds, benzoic acid (122 Da), testosterone (288 Da), naloxone (328 Da), and indomethacin (359 Da) through human skin. The permeability enhancement of each enhancer for each drug was calculated according to E.sub.c/pg=P.sub.e/pg/P.sub.pg, where P.sub.e/pg is the drug permeability from the enhancer/propylene glycol formulation and P.sub.pg is the permeability from propylene glycol alone.

The primary mechanism by which unsaturated fatty acids, such as linoleic acid, are thought to enhance skin permeability is by disordering the intercellular lipid domain. For example, detailed structural studies of unsaturated fatty acids, such as oleic acid, have been performed utilizing differential scanning calorimetry (Barry J. Controlled Release 6, 85-97 (1987)) and infrared spectroscopy (Ongpipattanankul, et al., Pharm. Res. 8, 350-354 (1991); Mark, et al., J. Control. Rd. 12, 67-75 (1990)). Oleic acid was found to disorder the highly ordered SC lipid bilayers, and to possibly form a separate, oil-like phase in the intercellular domain. SC Lipid bilayers disordered by unsaturated fatty acids or other bilayer disrupters may be similar in nature to fluid phase lipid bilayers.

A separated oil phase should have properties similar to a bulk oil phase. Much is known about transport a fluid bilayers and bulk oil phases. Specifically, diffusion coefficients in fluid phase, for example, dimyristoylphosphatidylcholine (DMPC) bilayers Clegg and Vaz In “Progress in Protein-Lipid Interactions” Watts, ed. (Elsevier, N.Y. 1985) 173-229; Tocanne, et al., FEB 257, 10-16 (1989) and in bulk oil phase Perry, et al., “Perry's Chemical Engineering Handbook” (McGraw-Hill, NY 1984) are greater than those in the SC, and more importantly, they exhibit size dependencies which are considerably weaker than that of SC transport Kasting, et al., In: “Prodrugs: Topical and Ocular Delivery” Sloan. ed. (Marcel Dekker, NY 1992) 117-161; Ports and Guy, Pharm. Res. 9, 663-339 (1992); Willschut, et al. Chemosphere 30, 1275-1296 (1995). As a result, the diffusion coefficient of a given solute will be greater in a fluid bilayer, such as DMPC, or a bulk oil phase than in the SC. Due to the strong size dependence of SC transport, diffusion in SC lipids is considerably slower for larger compounds, while transport in fluid DMPC bilayers and bulk oil phases is only moderately lower for larger compounds. The difference between the diffusion coefficient in the SC and those in fluid DMPC bilayers or bulk oil phases will be greater for larger solutes, and less for smaller compounds. Therefore, the enhancement ability of a bilayer disordering compound which can transform the SC lipids bilayers into a fluid bilayer phase or add a separate bulk oil phase should exhibit a size dependence, with smaller permeability enhancements for small compounds and larger enhancement for larger compounds.

Another way to increase the transdermal delivery of a drug is to use chemical solubility enhancers that increase the conjugated oligonucleotide solubility in its vehicle. This can be achieved either through changing drug-vehicle interaction by introducing different excipients, or through changing drug crystallinity (Flynn and Weiner, 1993). Solubility enhancers include water diols, such as propylene glycol and glycerol; mono-alcohols, such as ethanol, propanol, and higher alcohols; DMSO; dimethylformamide; N,N-dimethylacetamide; 2-pyrrolidone; N-(2-hydroxyethyl) pyrrolidone, N-methylpyrrolidone, 1-dodecylazacycloheptan-2-one and other n-substituted-alkyl-azacycloalkyl-2-ones.

In one embodiment, combinations of enhancers (Binary Systems) can be used with the conjugated oliogonucleotide. U.S. Pat. No. 4,537,776 to Cooper contains a summary of information detailing the use of certain binary systems for penetration enhancement. European Patent Application 43,738, also describes the use of selected diols as solvents along with a broad category of cell-envelope disordering compounds for delivery of lipophilic pharmacologically-active compounds. A binary system for enhancing metaclopramide penetration is disclosed in UK Patent Application GB 2,153,223 A, consisting of a monovalent alcohol ester of a C8-32 aliphatic monocarboxylic acid (unsaturated and/or branched if C18-32) or a C6-24 aliphatic monoalcohol (unsaturated and/or branched if C14-24) and an N-cyclic compound such as 2-pyrrolidone or N-methylpyrrolidone.

Combinations of enhancers consisting of diethylene glycol monoethyl or monomethyl ether with propylene glycol monolaurate and methyl laurate are disclosed in U.S. Pat. No. 4,973,468 for enhancing the transdermal delivery of steroids such as progestogens and estrogens. A dual enhancer consisting of glycerol monolaurate and ethanol for the transdermal delivery of drugs is described in U.S. Pat. No. 4,820,720. U.S. Pat. No. 5,006,342 lists numerous enhancers for transdermal drug administration consisting of fatty acid esters or fatty alcohol ethers of C.sub.2 to C.sub.4 alkanediols, where each fatty acid/alcohol portion of the ester/ether is of about 8 to 22 carbon atoms. U.S. Pat. No. 4,863,970 discloses penetration-enhancing compositions for topical application including an active permeant contained in a penetration-enhancing vehicle containing specified amounts of one or more cell-envelope disordering compounds such as oleic acid, oleyl alcohol, and glycerol esters of oleic acid; a C.sub.2 or C.sub.3 alkanol and an inert diluent such as water.

Other chemical enhancers, not necessarily associated with binary systems, include dimethylsulfoxide (DMSO) or aqueous solutions of DMSO such as those described in U.S. Pat. No. 3,551,554 to Herschler; U.S. Pat. No. 3,711,602 to Herschler and U.S. Pat. No. 3,711,606 to Herschler, and the azones (n-substituted-alkyl-azacycloalkyl-2-ones) such as noted in U.S. Pat. No. 4,557,943 to Cooper. In PCT/US96/12244 by Massachusetts Institute of Technology, passive experiments with polyethylene glycol 200 dilaurate (PEG), isopropyl myristate (IM), and glycerol trioleate (GT) result in corticosterone flux enhancement values of only 2, 5, and 0.8 relative to the passive flux from PBS alone. However, 50% ethanol and LA/ethanol significantly increase corticosterone passive fluxes by factors of 46 and 900.

Some chemical enhancer systems may possess negative side effects such as toxicity and skin irritations. U.S. Pat. No. 4,855,298 discloses compositions for reducing skin irritation caused by chemical enhancer-containing compositions having skin irritation properties with an amount of glycerin sufficient to provide an anti-irritating effect. The present invention enables testing of the effects of a large number of enhancers on tissue barrier transport, such as transdermal transport, of a compound, pharmaceutical, or other component.

In one embodiment, the conjugated oligonucleotide is coadmistered with ethanol. The amount of ethanol used is at least 0.3%, at least 0.5%, at least 1.0%, at least 2.0%, at least 2.5%, at least 3.0%, at least 4.0%, or at least 5.0%. Preferably the amount of ethanol used is between 0.5%-30%.

In one embodiment, the delivery of conjugated oligonucleotide can be further enhanced with the use of a mechanical enhancer. In one preferred embodiment, the mechanical enhancer is a Cytobrush®. The Cytobrush® of the invention can be a gentle or a full Cytobrush®.

Another aspect of the present invention provides a composition comprising one or more of conjugated oligonucleotide formulated for electroporation epithelial cells or muscle cells in vivo. For example, the conjugated oligonucleotide is formulated in supramolecular complexes or in liposomes.

Still another aspect of the present invention provides a method for delivering one or more conjugated oligonucleotide to a patient by electroporation, comprising administering the conjugated oligonucleotide of sufficient amount to an animal through electroporation, wherein the conjugated oligonucleotide attenuates expression of a target gene in cells of the patient. For example, the conjugated oligonucleotide of the method is formulated in supramolecular complexes or in liposomes.

In one embodiment, the invention provides a pharmaceutical preparation comprising at least one conjugated oligonucleotide formulated for electroporation into cells, and a pharmaceutically acceptable carrier. Optionally, the pharmaceutically acceptable carrier is selected from pharmaceutically acceptable salts, ester, and salts of such esters. In certain preferred embodiments, the invention provides a pharmaceutical package comprising the pharmaceutical preparation which includes at least one conjugated oligonucleotide formulated for electroporation into cells and a pharmaceutically acceptable carrier, in association with instructions (written and/or pictorial) for administering the preparation to a human patient.

The term “electroporation” as used herein refers to a method that utilized electric pulses to deliver a nucleic acid sequence into cells. In one example, electroporations is a technique for transferring nucleic acid (e.g., a nucleic acid containing a gene) into cells (e.g., plant cells), in which a DC high voltage pulse is applied to the cells to open pores allowing the nucleic acid to enter the cells. Conditions for electroporation may be appropriately selected by those skilled in the art, depending on the species, tissue, cells, or the like, which are used. A typical voltage used for electroporation is 10 V/cm to 200 V/cm, preferably 20 V/cm to 150 V/cm, more preferably 30 V/cm to 120 V/cm, even more preferably 40 V/cm to 100 V/cm, and most preferably 50 V/cm to 100 V/cm, but is not limited to these values. A typical pulse width for electroporation is 1 μsec to 90 μsec, preferably 10 μsec to 90 μsec, still preferably 20 μsec to 80 μsec, still more preferably 30 μsec to 80 μsec, still even more preferably 40 μsec to 70 μsec, and most preferably 50 μsec to 60 μsec, but is not limited to these values. A typical number of pulses for electroporation is 1 to 200, preferably 10 to 150, more preferably 20 to 120, even more preferably 30 to 110, and most preferably 40 to 100, but is not limited to these values.

The terms “electrical pulse” and “electroporation” as used herein refer to the administration of an electrical current to a tissue or cell for the purpose of taking up a nucleic acid molecule into a cell. A skilled artisan recognizes that these terms are associated with the terms “pulsed electric field” “pulsed current device” and “pulse voltage device.” A skilled artisan recognizes that the amount and duration of the electrical pulse is dependent on the tissue, size, and overall health of the recipient subject, and furthermore knows how to determine such parameters empirically.

In a further embodiment, mechanical enhancers are defined as including almost any extraneous enhancer, such as ultrasound, mechanical or osmotic pressure, electric fields (electroporation or iontophoresis) or magnetic fields.

There have been numerous reports on the use of ultrasound (typically in the range of 20 kHz to 10 MHz in frequency) to enhance transdermal delivery. Ultrasound has been applied alone and in combination with other chemical and/or mechanical enhancers. For example, as reported in PCT/US96/12244 by Massachusetts Institute of Technology, therapeutic ultrasound (1 MHz, 1.4 W/cm.sup.2) and the chemical enhancers utilized together produce corticosterone fluxes from PBS, PEG, 1M, and GT that are greater than the passive fluxes from the same enhancers by factors of between 1.3 and 5.0. Ultrasound combined with 50% ethanol produces a 2-fold increase in corticosterone transport above the passive case, but increase by 14-fold the transport from LA/Ethanol, yielding a flux of 0.16 mg/cm.sup.2/hr, 13.000-fold greater than that from PBS alone.

Pressure gradients can also be used to enhance movement of fluids across the skin. Pressure can be applied by a vacuum or a positive pressure device. Alternatively, osmotic pressure may be used to drive transdermal transport.

Similarly, application of an electric current has been shown to enhance transdermal drug transport and blood analyte extraction. Such electric current enhances transport by different mechanisms. For example, application of an electric field provides a driving force for the transport of charged molecules across the skin and second, ionic motion due to application of electric fields may induce convective flows across the skin, referred to as electro-osmosis. This mechanism is believed to play a dominant role in transdermal transport of neutral molecules during iontophoresis. Iontophoresis involves the application of an electrical current, preferably DC, or AC, at a current density of greater than zero up to about 1 mA/cm.sup.2. Enhancement of skin permeability using electric current to achieve transdermal extraction of glucose, was reported by Tamada, et al., Proceed. Intern. Symp. Control. Rel. Bioact. Mater. 22, 129-130 (1995).

Application of magnetic fields to the skin pretreated or in combination with other permeation enhancers can be used to transport magnetically active species across the skin. For example, polymer microspheres loaded with magnetic particles could be transported across the skin.

Another aspect of the present invention provides a composition comprising one or more of conjugated oligonucleotide formulated for intrademal injection, transdermal injection, or epidermal into epithelial tissues or muscle tissues. For example, the conjugated oligonucleotide is formulated in supramolecular complexes or in liposomes.

In one embodiment the conjugated oligonucleotide of the present invention is capable of: suppressing tumor growth, suppressing growth of papillomavirus-infected cells, e.g., HPV-infected cells; inhibiting growth of a papillomavirus-infected cell, e.g., an HPV-infected cell, e.g., a high-risk HPV infected cell, e.g., and HPV-16, -18, -31, or -33 infected cell, e.g., a bovine papillomavirus (BPV)-infected cell; inhibiting infection of a cell by a papillomavirus, e.g., an HPV, e.g., ahigh-risk HPV, e.g., and HPV-16, -18, -31, or -33, e.g., a bovine papillomavirus (BPV); inhibiting transformation of a cell by a papillomavirus, e.g., an HPV, e.g., a high-risk HPV, e.g., and HPV-16, -18, -31, or -33, e.g., a bovine papillomavirus; or inhibiting immortalization of a cell, e.g., a human cell, by a papillomavirus, e.g., an HPV, e.g., a high-risk HPV, e.g., and HPV-16, -18, -31, or -33, e.g., a bovine papillomavirus; inhibiting the growth of, or diminishing the size of a wart.

The term “aliphatic” refers to non-aromatic moiety that may contain any combination of carbon atoms, hydrogen atoms, halogen atoms, oxygen, nitrogen or other atoms, and optionally contain one or more units of unsaturation, e.g., double and/or triple bonds. An aliphatic group may be straight chained, branched or cyclic and preferably contains between about 1 and about 24 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted.

“Alkyl” means a straight chain or branched, noncyclic or cyclic, saturated aliphatic hydrocarbon containing from 1 to 24 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like.

“Alkenyl” means an alkyl, as defined above, containing at least one double bond between adjacent carbon atoms. Alkenyls include both cis and trans isomers. Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like.

“Alkynyl” means any alkyl or alkenyl, as defined above, which additionally contains at least one triple bond between adjacent carbons. Representative straight chain and branched alkynyls include acetylenyl, propynyl, 1-butynyl, 2-butynyl, 1-pentynyl, 2-pentynyl, 3-methyl-1 butynyl, and the like.

The compounds of the present invention may be prepared by known organic synthesis techniques, including the methods described in more detail in the Examples.

In the context of this invention, the term “oligonucleotide” refers to an oligomer or polymer of ribonucleic acid (RNA), deoxyribonucleic acid (DNA), or modified versions thereof, or RNA or DNA mimetics, or combinations thereof. This term, therefore, includes oligonucleotides composed of naturally-occurring nucleobases, sugars and covalent internucleoside (backbone) linkages as well as oligonucleotides having non-naturally-occurring portions, which function similarly. Such modified or substituted oligonucleotides are often preferred over native forms because of desirable properties such as, for example, enhanced cellular uptake, enhanced affinity for the nucleic acid target and increased stability in the presence of nucleases. The oligonucleotides according to the present invention can be single-stranded or they can be double-stranded. Oligonucleotides of the invention include, but not limited to siRNA, microRNA, antagomirs, antisense, and ribozyme.

RNA Interference Nucleic Acids

In particular embodiments, lipophilic conjugated oligonucleotides of the present invention are associated with RNA interference (RNAi) molecules. RNA interference methods using RNAi molecules may be used to disrupt the expression of a gene or polynucleotide of interest. In the last 5 years small interfering RNA (siRNA) has essentially replaced antisense ODN and ribozymes as the next generation of targeted oligonucleotide drugs under development. SiRNAs are RNA duplexes normally 21-30 nucleotides long that can associate with a cytoplasmic multi-protein complex known as RNAi-induced silencing complex (RISC). RISC loaded with siRNA mediates the degradation of homologous mRNA transcripts, therefore siRNA can be designed to knock down protein expression with high specificity. Unlike other antisense technologies, siRNA function through a natural mechanism evolved to control gene expression through non-coding RNA. This is generally considered to be the reason why their activity is more potent in vitro and in vivo than either antisense ODN or ribozymes. A variety of RNAi reagents, including siRNAs targeting clinically relevant targets, are currently under pharmaceutical development, as described, e.g., in de Fougerolles, A. et al., Nature Reviews 6:443-453 (2007).

While the first described RNAi molecules were RNA:RNA hybrids comprising both an RNA sense and an RNA antisense strand, it has now been demonstrated that DNA sense:RNA antisense hybrids, RNA sense:DNA antisense hybrids, and DNA:DNA hybrids are capable of mediating RNAi (Lamberton, J. S. and Christian, A. T., (2003) Molecular Biotechnology 24:111-119). Thus, the invention includes the use of RNAi molecules comprising any of these different types of double-stranded molecules. In addition, it is understood that RNAi molecules may be used and introduced to cells in a variety of forms. Accordingly, as used herein, RNAi molecules encompasses any and all molecules capable of inducing an RNAi response in cells, including, but not limited to, double-stranded polynucleotides comprising two separate strands, i.e. a sense strand and an antisense strand, e.g., small interfering RNA (siRNA); polynucleotides comprising a hairpin loop of complementary sequences, which forms a double-stranded region, e.g., shRNAi molecules, and expression vectors that express one or more polynucleotides capable of forming a double-stranded polynucleotide alone or in combination with another polynucleotide.

RNA interference (RNAi) may be used to specifically inhibit expression of target polynucleotides. Double-stranded RNA-mediated suppression of gene and nucleic acid expression may be accomplished according to the invention by introducing dsRNA, siRNA or shRNA into cells or organisms. SiRNA may be double-stranded RNA, or a hybrid molecule comprising both RNA and DNA, e.g., one RNA strand and one DNA strand. It has been demonstrated that the direct introduction of siRNAs to a cell can trigger RNAi in mammalian cells (Elshabir, S. M., et al. Nature 411:494-498 (2001)). Furthermore, suppression in mammalian cells occurred at the RNA level and was specific for the targeted genes, with a strong correlation between RNA and protein suppression (Caplen, N. et al., Proc. Natl. Acad. Sci. USA 98:9746-9747 (2001)). In addition, it was shown that a wide variety of cell lines, including HeLa S3, COS7, 293, NIH/3T3, A549, HT-29, CHO-KI and MCF-7 cells, are susceptible to some level of siRNA silencing (Brown, D. et al. TechNotes 9(1):1-7, available at http://www.dot.ambion.dot.com/techlib/tn/91/912.html (Sep. 1, 2002)).

RNAi molecules targeting specific polynucleotides can be readily prepared according to procedures known in the art. Structural characteristics of effective siRNA molecules have been identified. Elshabir, S. M. et al. (2001) Nature 411:494-498 and Elshabir, S. M. et al. (2001), EMBO 20:6877-6888. Accordingly, one of skill in the art would understand that a wide variety of different siRNA molecules may be used to target a specific gene or transcript. In certain embodiments, siRNA molecules according to the invention are double-stranded and 16-30 or 18-25 nucleotides in length, including each integer in between. In one embodiment, an siRNA is 21 nucleotides in length. In certain embodiments, siRNAs have 0-7 nucleotide 3′ overhangs or 0-4 nucleotide 5′ overhangs. In one embodiment, an siRNA molecule has a two nucleotide 3′ overhang. In one embodiment, an siRNA is 21 nucleotides in length with two nucleotide 3′ overhangs (i.e. they contain a 19 nucleotide complementary region between the sense and antisense strands). In certain embodiments, the overhangs are UU or dTdT 3′ overhangs.

Generally, siRNA molecules are completely complementary to the target mRNA molecule, since even single base pair mismatches have been shown to reduce silencing. In other embodiments, siRNAs may have a modified backbone composition, such as, for example, 2′-deoxy- or 2′-O-methyl modifications. However, in preferred embodiments, the entire strand of the siRNA is not made with either 2′ deoxy or 2′-O-modified bases.

In one embodiment, siRNA target sites are selected by scanning the target mRNA transcript sequence for the occurrence of AA dinucleotide sequences. Each AA dinucleotide sequence in combination with the 3′ adjacent approximately 19 nucleotides are potential siRNA target sites. In one embodiment, siRNA target sites are preferentially not located within the 5′ and 3′ untranslated regions (UTRs) or regions near the start codon (within approximately 75 bases), since proteins that bind regulatory regions may interfere with the binding of the siRNP endonuclease complex (Elshabir, S. et al. Nature 411:494-498 (2001); Elshabir, S. et al. EMBO J. 20:6877-6888 (2001)). In addition, potential target sites may be compared to an appropriate genome database, such as BLASTN 2.0.5, available on the NCBI server at www.ncbi.nlm, and potential target sequences with significant homology to other coding sequences eliminated.

In one embodiment, lipophilic conjugated oligonucleotides of the invention includes short hairpin RNAs. Short Hairpin RNA (shRNA) is a form of hairpin RNA capable of sequence-specifically reducing expression of a target gene. Short hairpin RNAs may offer an advantage over siRNAs in suppressing gene expression, as they are generally more stable and less susceptible to degradation in the cellular environment. It has been established that such short hairpin RNA-mediated gene silencing works in a variety of normal and cancer cell lines, and in mammalian cells, including mouse and human cells. Paddison, P. et al., Genes Dev. 16(8):948-58 (2002). Furthermore, transgenic cell lines bearing chromosomal genes that code for engineered shRNAs have been generated. These cells are able to constitutively synthesize shRNAs, thereby facilitating long-lasting or constitutive gene silencing that may be passed on to progeny cells. Paddison, P. et al., Proc. Natl. Acad. Sci. USA 99(3):1443-1448 (2002).

ShRNAs contain a stem loop structure. In certain embodiments, they may contain variable stem lengths, typically from 19 to 29 nucleotides in length, or any number in between. In certain embodiments, hairpins contain 19 to 21 nucleotide stems, while in other embodiments, hairpins contain 27 to 29 nucleotide stems. In certain embodiments, loop size is between 4 to 23 nucleotides in length, although the loop size may be larger than 23 nucleotides without significantly affecting silencing activity. ShRNA molecules may contain mismatches, for example G-U mismatches between the two strands of the shRNA stem without decreasing potency. In fact, in certain embodiments, shRNAs are designed to include one or several G-U pairings in the hairpin stem to stabilize hairpins during propagation in bacteria, for example. However, complementarity between the portion of the stem that binds to the target mRNA (antisense strand) and the mRNA is typically required, and even a single base pair mismatch is this region may abolish silencing. 5′ and 3′ overhangs are not required, since they do not appear to be critical for shRNA function, although they may be present (Paddison et al. (2002) Genes & Dev. 16(8):948-58).

MicroRNAs

In one embodiment, a nucleic acid is a Micro RNA (miRNA), MicroRNA mimic or an antagomir. Micro RNAs (miRNAs) are a highly conserved class of small RNA molecules that are transcribed from DNA in the genomes of plants and animals, but are not translated into protein. Processed miRNAs are single stranded ˜17-25 nucleotide (nt) RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. RISC mediates down-regulation of gene expression through translational inhibition, transcript cleavage, or both. RISC is also implicated in transcriptional silencing in the nucleus of a wide range of eukaryotes.

The number of miRNA sequences identified to date is large and growing, illustrative examples of which can be found, for example, in: “miRBase: microRNA sequences, targets and gene nomenclature” Griffiths-Jones S, Grocock R J, van Dongen S, Bateman A, Enright A J. NAR, 2006, 34, Database Issue, D140-D144; “The microRNA Registry” Griffiths-Jones S, NAR, 2004, 32, Database Issue, D109-D111; and also at http://microrna.sanger.ac.uk/sequences/.

Antisense Oligonucleotides

In one embodiment, a nucleic acid is an antisense oligonucleotide directed to a target polynucleotide. The term “antisense oligonucleotide” or simply “antisense” is meant to include oligonucleotides that are complementary to a targeted polynucleotide sequence. Antisense oligonucleotides are single strands of DNA or RNA that are complementary to a chosen sequence. In the case of antisense RNA, they prevent translation of complementary RNA strands by binding to it. Antisense DNA can be used to target a specific, complementary (coding or non-coding) RNA. If binding takes places this DNA/RNA hybrid can be degraded by the enzyme RNase H. In particular embodiment, antisense oligonucleotides contain from about 10 to about 50 nucleotides, more preferably about 15 to about 30 nucleotides. The term also encompasses antisense oligonucleotides that may not be exactly complementary to the desired target gene. Thus, the invention can be utilized in instances where non-target specific-activities are found with antisense, or where an antisense sequence containing one or more mismatches with the target sequence is the most preferred for a particular use.

Antisense oligonucleotides have been demonstrated to be effective and targeted inhibitors of protein synthesis, and, consequently, can be used to specifically inhibit protein synthesis by a targeted gene. The efficacy of antisense oligonucleotides for inhibiting protein synthesis is well established. For example, the synthesis of polygalactauronase and the muscarine type 2 acetylcholine receptor are inhibited by antisense oligonucleotides directed to their respective mRNA sequences (U.S. Pat. No. 5,739,119 and U.S. Pat. No. 5,759,829). Further, examples of antisense inhibition have been demonstrated with the nuclear protein cyclin, the multiple drug resistance gene (MDG1), ICAM-1, E-selectin, STK-1, striatal GABA_{A} receptor and human EGF (Jaskulski et al., Science. 1988 Jun. 10; 240(4858):1544-6; Vasanthakumar and Ahmed, Cancer Commun. 1989; 1(4):225-32; Penis et al., Brain Res Mol Brain Res. 1998 Jun. 15; 57(2):310-20; U.S. Pat. No. 5,801,154; U.S. Pat. No. 5,789,573; U.S. Pat. No. 5,718,709 and U.S. Pat. No. 5,610,288). Furthermore, antisense constructs have also been described that inhibit and can be used to treat a variety of abnormal cellular proliferations, e.g. cancer (U.S. Pat. No. 5,747,470; U.S. Pat. No. 5,591,317 and U.S. Pat. No. 5,783,683).

Methods of producing antisense oligonucleotides are known in the art and can be readily adapted to produce an antisense oligonucleotide that targets any polynucleotide sequence. Selection of antisense oligonucleotide sequences specific for a given target sequence is based upon analysis of the chosen target sequence and determination of secondary structure, Tₘ, binding energy, and relative stability. Antisense oligonucleotides may be selected based upon their relative inability to form dimers, hairpins, or other secondary structures that would reduce or prohibit specific binding to the target mRNA in a host cell. Highly preferred target regions of the mRNA include those regions at or near the AUG translation initiation codon and those sequences that are substantially complementary to 5′ regions of the mRNA. These secondary structure analyses and target site selection considerations can be performed, for example, using v.4 of the OLIGO primer analysis software (Molecular Biology Insights) and/or the BLASTN 2.0.5 algorithm software (Altschul et al., Nucleic Acids Res. 1997, 25(17):3389-402).

Ribozymes

According to another embodiment of the invention, a nucleic acid is a ribozyme. Ribozymes are RNA-protein complexes having specific catalytic domains that possess endonuclease activity (Kim and Cech, Proc Natl Acad Sci USA. 1987 December; 84(24):8788-92; Forster and Symons, Cell. 1987 Apr. 24; 49(2):211-20). For example, a large number of ribozymes accelerate phosphodiester transfer reactions with a high degree of specificity, often cleaving only one of several phosphodiesters in an oligonucleotide substrate (Cech et al., Cell. 1981 December; 27(3 Pt 2):487-96; Michel and Westhof, J Mol. Biol. 1990 Dec. 5; 216(3):585-610; Reinhold-Hurek and Shub, Nature. 1992 May 14; 357(6374):173-6). This specificity has been attributed to the requirement that the substrate bind via specific base-pairing interactions to the internal guide sequence (“IGS”) of the ribozyme prior to chemical reaction.

At least six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nucleic acid molecule may be formed in a hammerhead, hairpin, a hepatitis δ virus, group I intron or RNaseP RNA (in association with an RNA guide sequence) or Neurospora VS RNA motif, for example. Specific examples of hammerhead motifs are described by Rossi et al. Nucleic Acids Res. 1992 Sep. 11; 20(17):4559-65. Examples of hairpin motifs are described by Hampel et al. (Eur. Pat. Appl. Publ. No. EP 0360257), Hampel and Tritz, Biochemistry 1989 Jun. 13; 28(12):4929-33; Hampel et al., Nucleic Acids Res. 1990 Jan. 25; 18(2):299-304 and U.S. Pat. No. 5,631,359. An example of the hepatitis δ virus motif is described by Perrotta and Been, Biochemistry. 1992 Dec. 1; 31(47):11843-52; an example of the RNaseP motif is described by Guerrier-Takada et al., Cell. 1983 December; 35(3 Pt 2):849-57; Neurospora VS RNA ribozyme motif is described by Collins (Saville and Collins, Cell. 1990 May 18; 61(4):685-96; Saville and Collins, Proc Natl Acad Sci USA. 1991 Oct. 1; 88(19):8826-30; Collins and Olive, Biochemistry. 1993 Mar. 23; 32(11):2795-9); and an example of the Group I intron is described in U.S. Pat. No. 4,987,071. Desirable characteristics of enzymatic nucleic acid molecules used according to the invention are that they have a specific substrate binding site which is complementary to one or more of the target RNA regions, and that they have nucleotide sequences within or surrounding that substrate binding site which impart an RNA cleaving activity to the molecule. Thus the ribozyme constructs need not be limited to specific motifs mentioned herein.

Methods of producing a ribozyme targeted to any polynucleotide sequence are known in the art. Ribozymes may be designed as described in Int. Pat. Appl. Publ. No. WO 93/23569 and Int. Pat. Appl. Publ. No. WO 94/02595, each specifically incorporated herein by reference, and synthesized to be tested in vitro and in vivo, as described therein.

Ribozyme activity can be optimized by altering the length of the ribozyme binding arms or chemically synthesizing ribozymes with modifications that prevent their degradation by serum ribonucleases (see e.g., Int. Pat. Appl. Publ. No. WO 92/07065; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

In one embodiment, the formulations of the invention can be used to silence or modulate a target gene such as but not limited to K14, E6AP, ENaC, Lamin A/C, FVII, Eg5, PCSK9, TPX2, apoB, SAA, TTR, RSV, PDGF beta gene, Erb-B gene, Src gene, CRK gene, GRB2 gene, RAS gene, MEKK gene, JNK gene, RAF gene, Erk1/2 gene, PCNA(p21) gene, MYB gene, JUN gene, FOS gene, BCL-2 gene, Cyclin D gene, VEGF gene, EGFR gene, Cyclin A gene, Cyclin E gene, WNT-1 gene, beta-catenin gene, c-MET gene, PKC gene, NFKB gene, STAT3 gene, survivin gene, Her2/Neu gene, topoisomerase I gene, topoisomerase II alpha gene, mutations in the p73 gene, mutations in the p21(WAF1/CIP1) gene, mutations in the p27(KIP1) gene, mutations in the PPM1D gene, mutations in the RAS gene, mutations in the caveolin I gene, mutations in the MIB I gene, mutations in the MTAI gene, mutations in the M68 gene, mutations in tumor suppressor genes, mutations in the p53 tumor suppressor gene, mutations in the p53 family member DN-p63, mutations in the pRb tumor suppressor gene, mutations in the APC1 tumor suppressor gene, mutations in the BRCA1 tumor suppressor gene, mutations in the PTEN tumor suppressor gene, mLL fusion gene, BCR/ABL fusion gene, TEL/AML1 fusion gene, EWS/FLI1 fusion gene, TLS/FUS1 fusion gene, PAX3/FKHR fusion gene, AML1/ETO fusion gene, alpha v-integrin gene, Flt-1 receptor gene, tubulin gene, Human Papilloma Virus gene, a gene required for Human Papilloma Virus replication, Human Immunodeficiency Virus gene, a gene required for Human Immunodeficiency Virus replication, Hepatitis A Virus gene, a gene required for Hepatitis A Virus replication, Hepatitis B Virus gene, a gene required for Hepatitis B Virus replication, Hepatitis C Virus gene, a gene required for Hepatitis C Virus replication, Hepatitis D Virus gene, a gene required for Hepatitis D Virus replication, Hepatitis E Virus gene, a gene required for Hepatitis E Virus replication, Hepatitis F Virus gene, a gene required for Hepatitis F Virus replication, Hepatitis G Virus gene, a gene required for Hepatitis G Virus replication, Hepatitis H Virus gene, a gene required for Hepatitis H Virus replication, Respiratory Syncytial Virus gene, a gene that is required for Respiratory Syncytial Virus replication, Herpes Simplex Virus gene, a gene that is required for Herpes Simplex Virus replication, herpes Cytomegalovirus gene, a gene that is required for herpes Cytomegalovirus replication, herpes Epstein Barr Virus gene, a gene that is required for herpes Epstein Barr Virus replication, Kaposi's Sarcoma-associated Herpes Virus gene, a gene that is required for Kaposi's Sarcoma-associated Herpes Virus replication, JC Virus gene, human gene that is required for JC Virus replication, myxovirus gene, a gene that is required for myxovirus gene replication, rhinovirus gene, a gene that is required for rhinovirus replication, coronavirus gene, a gene that is required for coronavirus replication, West Nile Virus gene, a gene that is required for West Nile Virus replication, St. Louis Encephalitis gene, a gene that is required for St. Louis Encephalitis replication, Tick-borne encephalitis virus gene, a gene that is required for Tick-borne encephalitis virus replication, Murray Valley encephalitis virus gene, a gene that is required for Murray Valley encephalitis virus replication, dengue virus gene, a gene that is required for dengue virus gene replication, Simian Virus 40 gene, a gene that is required for Simian Virus 40 replication, Human T Cell Lymphotropic Virus gene, a gene that is required for Human T Cell Lymphotropic Virus replication, Moloney-Murine Leukemia Virus gene, a gene that is required for Moloney-Murine Leukemia Virus replication, encephalomyocarditis virus gene, a gene that is required for encephalomyocarditis virus replication, measles virus gene, a gene that is required for measles virus replication, Vericella zoster virus gene, a gene that is required for Vericella zoster virus replication, adenovirus gene, a gene that is required for adenovirus replication, yellow fever virus gene, a gene that is required for yellow fever virus replication, poliovirus gene, a gene that is required for poliovirus replication, poxvirus gene, a gene that is required for poxvirus replication, plasmodium gene, a gene that is required for plasmodium gene replication, Mycobacterium ulcerans gene, a gene that is required for Mycobacterium ulcerans replication, Mycobacterium tuberculosis gene, a gene that is required for Mycobacterium tuberculosis replication, Mycobacterium leprae gene, a gene that is required for Mycobacterium leprae replication, Staphylococcus aureus gene, a gene that is required for Staphylococcus aureus replication, Streptococcus pneumoniae gene, a gene that is required for Streptococcus pneumoniae replication, Streptococcus pyogenes gene, a gene that is required for Streptococcus pyogenes replication, Chlamydia pneumoniae gene, a gene that is required for Chlamydia pneumoniae replication, Mycoplasma pneumoniae gene, a gene that is required for Mycoplasma pneumoniae replication, an integrin gene, a selectin gene, complement system gene, chemokine gene, chemokine receptor gene, GCSF gene, Gro 1 gene, Gro2 gene, Gro3 gene, PF4 gene, MIG gene, Pro-Platelet Basic Protein gene, MIP-1I gene, MIP-1J gene, RANTES gene, MCP-1 gene, MCP-2 gene, MCP-3 gene, CMBKR1 gene, CMBKR2 gene, CMBKR3 gene, CMBKR5v, AIF-1 gene, 1-309 gene, a gene to a component of an ion channel, a gene to a neurotransmitter receptor, a gene to a neurotransmitter ligand, amyloid-family gene, presenilin gene, HD gene, DRPLA gene, SCA1 gene, SCA2 gene, MJD1 gene, CACNL1A4 gene, SCAT gene, SCA8 gene, allele gene found in LOH cells, or one allele gene of a polymorphic gene.

Immunostimulatory Oligonucleotides

The lipohilic conjugated oligonucleotides of the present invention may be immunostimulatory, including immunostimulatory oligonucleotides (ISS; single- or double-stranded) capable of inducing an immune response when administered to a subject, which may be a mammal or other patient. ISS include, e.g., certain palindromes leading to hairpin secondary structures (see Yamamoto S., et al. (1992) J. Immunol. 148: 4072-4076), or CpG motifs, as well as other known ISS features (such as multi-G domains, see WO 96/11266).

The immune response may be an innate or an adaptive immune response. The immune system is divided into a more innate immune system, and acquired adaptive immune system of vertebrates, the latter of which is further divided into humoral cellular components. In particular embodiments, the immune response may be mucosal.

Immunostimulatory nucleic acids are considered to be non-sequence specific when it is not required that they specifically bind to and reduce the expression of a target polynucleotide in order to provoke an immune response. Thus, certain immunostimulatory nucleic acids may comprise a sequence corresponding to a region of a naturally occurring gene or mRNA, but they may still be considered non-sequence specific immunostimulatory nucleic acids.

In one embodiment, the immunostimulatory nucleic acid or oligonucleotide comprises at least one CpG dinucleotide. The oligonucleotide or CpG dinucleotide may be unmethylated or methylated. In another embodiment, the immunostimulatory nucleic acid comprises at least one CpG dinucleotide having a methylated cytosine. In one embodiment, the nucleic acid comprises a single CpG dinucleotide, wherein the cytosine in said CpG dinucleotide is methylated. In a specific embodiment, the nucleic acid comprises the sequence 5′ TAACGTTGAGGGGCAT 3′. In an alternative embodiment, the nucleic acid comprises at least two CpG dinucleotides, wherein at least one cytosine in the CpG dinucleotides is methylated. In a further embodiment, each cytosine in the CpG dinucleotides present in the sequence is methylated. In another embodiment, the nucleic acid comprises a plurality of CpG dinucleotides, wherein at least one of said CpG dinucleotides comprises a methylated cytosine.

In one specific embodiment, the nucleic acid comprises the sequence 5′ TTCCATGACGTTCCTGACGT 3′. In another specific embodiment, the nucleic acid sequence comprises the sequence 5′ TCCATGACGTTCCTGACGT 3′, wherein the two cytosines indicated in bold are methylated. In particular embodiments, the ODN is selected from a group of ODNs consisting of ODN #1, ODN #2, ODN #3, ODN #4, ODN #5, ODN #6, ODN #7, ODN #8, and ODN #9, as shown below.

TABLE 1
Exemplary Immunostimulatory Oligonucleotides (ODNs)

| | ODN | ODN |
|---|---|---|
| ODN NAME | SEQ ID NO | SEQUENCE (5′-3′). |
| ODN 1 | | 5′-TAACGTTGAGGGGCAT-3′ |
| human c-myc | | |
| * ODN 1m | | 5′-TAAZGTTGAGGGGCAT-3′ |
| ODN 2 | | 5′-TCCATGACGTTCCTGACGTT-3′ |
| * ODN 2m | | 5′-TCCATGAZGTTCCTGAZGTT-3′ |
| ODN 3 | | 5′-TAAGCATACGGGGTGT-3′ |
| ODN 5 | | 5′-AACGTT-3′ |
| ODN 6 | | 5′-GATGCTGTGTCGGGGTCTCCGGGC-3′ |
| ODN 7 | | 5′-TCGTCGTTTTGTCGTTTTGTCGTT-3′ |
| ODN 7m | | 5′-TZGTZGTTTTGTZGTTTTGTZGTT-3′ |
| ODN 8 | | 5′-TCCAGGACTTCTCTCAGGTT-3′ |
| ODN 9 | | 5′-TCTCCCAGCGTGCGCCAT-3′ |
| ODN 10 murine | | 5′-TGCATCCCCCAGGCCACCAT-3′ |
| Intracellular | | |
| Adhesion Molecule-1 | | |
| ODN 11 human | | 5′-GCCCAAGCTGGCATCCGTCA-3′ |
| Intracellular | | |
| Adhesion Molecule-1 | | |
| ODN 12 human | | 5′-GCCCAAGCTGGCATCCGTCA-3′ |
| Intracellular | | |
| Adhesion Molecule-1 | | |
| ODN 13 human | | 5′-GGT GCTCACTGC GGC-3′ |
| erb-B-2 | | |
| ODN 14 human | | 5′-AACC GTT GAG GGG CAT-3′ |
| c-myc | | |
| ODN 15 human | | 5′-TAT GCT GTG CCG GGG |
| c-myc | | TCT TCG GGC-3′ |
| ODN 16 | | 5′-GTGCCG GGGTCTTCGGGC-3′ |
| ODN 17 human | | 5′-GGACCCTCCTCCGGAGCC-3′ |
| Insulin Growth | | |
| Factor 1-Receptor | | |
| ODN 18 human | | 5'-TCC TCC GGA GCC AGA CTT-3′ |
| Insulin Growth | | |
| Factor 1-Receptor | | |
| ODN 19 human | | 5′-AAC GTT GAG GGG CAT-3′ |
| Epidermal Growth | | |
| Factor-Receptor | | |
| ODN 20 | | 5′-CCGTGGTCA TGCTCC-3′ |
| Epidermal Growth | | |
| Factor-Receptor | | |
| ODN 21 human | | 5′-CAG CCTGGCTCACCG CCTTGG-3′ |
| Vascular Endothelial | | |
| Growth Factor | | |
| ODN 22 murine | | 5′-CAG CCA TGG TTC CCC CCA AC-3′ |
| Phosphokinase C-alpha | | |
| ODN 23 | | 5′-GTT CTC GCT GGT GAG TTT CA-3′ |
| ODN 24 human | | 5′-TCT CCCAGCGTGCGCCAT-3′ |
| Bcl-2 | | |
| ODN 25 human | | 5′-GTG CTC CAT TGA TGC-3′ |
| C-Raf-s | | |
| ODN #26 human | | 5′-GAGUUCUGAUGAGGCCGAAAGG |
| Vascular | | CCGAAAGUCUG-3′ |
| Endothelial Growth | | |
| Factor Receptor-1 | | |
| ODN #27 | | 5′-RRCGYY-3′ |
| ODN #28 | | 5′-AACGTTGAGGGGCAT-3′ |
| ODN #29 | | 5′-CAACGTTATGGGGAGA-3′ |
| ODN #30 human | | 5′-TAACGTTGAGGGGCAT-3′ |
| c-myc | | |

“Z” represents a methylated cytosine residue.
Note:
ODN 14 is a 15-mer oligonucleotide and ODN 1 is the same oligonucleotide
having a thymidine added onto the 5' end making ODN 1 into a 16-mer. No
difference in biological activity between ODN 14 and ODN 1 has been detected and
both exhibit similar immunostimulatory activity (Mui et al., 2001)

Additional specific nucleic acid sequences of oligonucleotides (ODNs) suitable for use in the compositions and methods of the invention are described in Raney et al., Journal of Pharmacology and Experimental Therapeutics, 298:1185-1192 (2001). In certain embodiments, ODNs used in the compositions and methods of the present invention have a phosphodiester (“PO”) backbone or a phosphorothioate (“PS”) backbone, and/or at least one methylated cytosine residue in a CpG motif.

Nucleic Acid Modifications

In the 1990's DNA-based antisense oligodeoxynucleotides (ODN) and ribozymes (RNA) represented an exciting new paradigm for drug design and development, but their application in vivo was prevented by endo- and exo-nuclease activity as well as a lack of successful intracellular delivery. The degradation issue was effectively overcome following extensive research into chemical modifications that prevented the oligonucleotide (oligo) drugs from being recognized by nuclease enzymes but did not inhibit their mechanism of action. This research was so successful that antisense ODN drugs in development today remain intact in vivo for days compared to minutes for unmodified molecules. (Kurreck, J. 2003. Antisense technologies. Improvement through novel chemical modifications. Eur J Biochem 270:1628-44). However, intracellular delivery and mechanism of action issues have so far limited antisense ODN and ribozymes from becoming clinical products.

RNA duplexes are inherently more stable to nucleases than single stranded DNA or RNA, and unlike antisense ODN, unmodified siRNA show good activity once they access the cytoplasm. Even so, the chemical modifications developed to stabilize antisense ODN and ribozymes have also been systematically applied to siRNA to determine how much chemical modification can be tolerated and if pharmacokinetic and pharmacodynamic activity can be enhanced. RNA interference by siRNA duplexes requires an antisense and sense strand, which have different functions. Both are necessary to enable the siRNA to enter RISC, but once loaded the two strands separate and the sense strand is degraded whereas the antisense strand remains to guide RISC to the target mRNA. Entry into RISC is a process that is structurally less stringent than the recognition and cleavage of the target mRNA. Consequently, many different chemical modifications of the sense strand are possible, but only limited changes are tolerated by the antisense strand (Zhang et al., 2006).

As is known in the art, a nucleoside is a base-sugar combination. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

### a. Backbone Modifications

Antisense, siRNA and other oligonucleotides useful in this invention include, but are not limited to, oligonucleotides containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone can also be considered to be oligonucleosides. Modified oligonucleotide backbones include, for example, phosphorothioates, chiral phosphorothioates, phosphorodithioates, phosphotriesters, aminoalkylphosphotri-esters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, phosphoroselenate, methylphosphonate, or O-alkyl phosphotriester linkages, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Particular non-limiting examples of particular modifications that may be present in a nucleic acid according to the present invention are shown in Table 2.

Various salts, mixed salts and free acid forms are also included. Representative United States patents that teach the preparation of the above linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

In certain embodiments, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include, e.g., those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that describe the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

The phosphorothioate backbone modification (Table 2, #1), where a non-bridging oxygen in the phosphodiester bond is replaced by sulfur, is one of the earliest and most common means deployed to stabilize nucleic acid drugs against nuclease degradation. In general, it appears that PS modifications can be made extensively to both siRNA strands without much impact on activity (Kurreck, J., Eur. J. Biochem. 270:1628-44, 2003). However, PS oligos are known to avidly associate nonspecifically with proteins resulting in toxicity, especially upon i.v. administration. Therefore, the PS modification is usually restricted to one or two bases at the 3′ and 5′ ends. The boranophosphate linker (Table 2, #2) is a recent modification that is apparently more stable than PS, enhances siRNA activity and has low toxicity (Hall et al., Nucleic Acids Res. 32:5991-6000, 2004).

TABLE 2
Chemical Modifications Applied to siRNA and Other Nucleic Acids

| | | | Modification | |
|---|---|---|---|---|
| # | Abbreviation | Name | Site | Structure |
| 1 | PS | Phosphorothioate | Backbone | 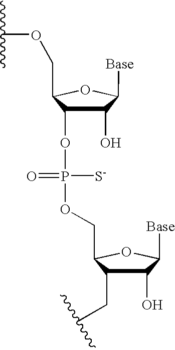 |
| 2 | PB | Boranophosphate | Backbone | 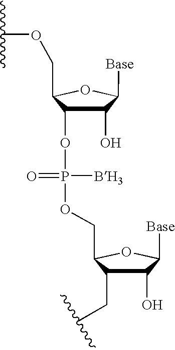 |
| 3 | N3-MU | N3-methyl-uridine | Base | 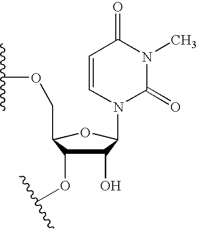 |
| 4 | 5′-BU | 5′-bromo-uracil | Base | 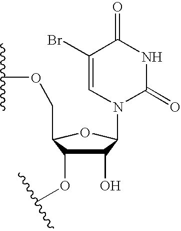 |
| 5 | 5′-IU | 5′-iodo-uracil | Base | 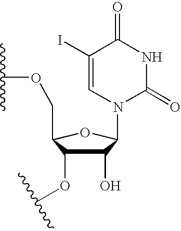 |
| 6 | 2,6-DP | 2,6- diaminopurine | Base | 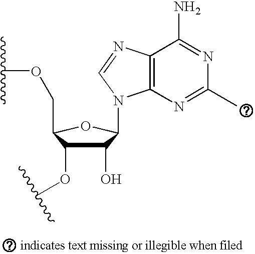 |
| 7 | 2′-F | 2′-Fluoro | Sugar | 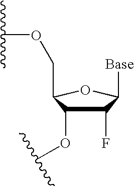 |
| 8 | 2′-OME | 2″-O-methyl | Sugar | 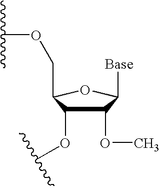 |
| 9 | 2′-O—MOE | 2′-O-(2- methoxylethyl) | Sugar | 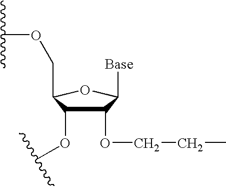 |
| 10 | 2′-DNP | 2′-O-(2,4- dinitrophenyl) | Sugar | 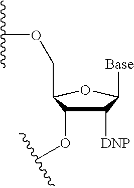 |
| 11 | LNA | Locked Nucleic Acid (methylene bridge connecting the 2′- oxygen with the 4′-carbon of the ribose ring) | Sugar | 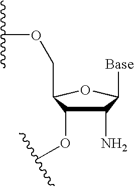 |
| 12 | 2′- Amino | 2′-Amino | Sugar | 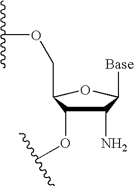 |
| 13 | 2′- Deoxy | 2′-Deoxy | Sugar | 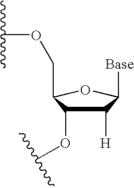 |
| 14 | 4′-thio | 4′-thio- ribonucleotide | Sugar | 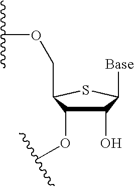 |

Other useful nucleic acids derivatives include those nucleic acids molecules in which the bridging oxygen atoms (those forming the phosphoester linkages) have been replaced with —S—, —NH—, —CH2- and the like. In certain embodiments, the alterations to the antisense, siRNA, or other nucleic acids used will not completely affect the negative charges associated with the nucleic acids. Thus, the present invention contemplates the use of antisense, siRNA, and other nucleic acids in which a portion of the linkages are replaced with, for example, the neutral methyl phosphonate or phosphoramidate linkages. When neutral linkages are used, in certain embodiments, less than 80% of the nucleic acid linkages are so substituted, or less than 50% of the linkages are so substituted.

### b. Base Modifications

Base modifications are less common than those to the backbone and sugar. The modifications shown in 0.3-6 all appear to stabilize siRNA against nucleases and have little effect on activity (Zhang, H. Y., Du, Q., Wahlestedt, C., Liang, Z. 2006. RNA Interference with chemically modified siRNA. Curr Top Med Chem 6:893-900).

Accordingly, oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C or m5c), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

Certain nucleobases are particularly useful for increasing the binding affinity of the oligomeric compounds of the invention, including 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications 1993, CRC Press, Boca Raton, pages 276-278). These may be combined, in particular embodiments, with 2′-O-methoxyethyl sugar modifications. United States patents that teach the preparation of certain of these modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941.

### c. Sugar Modifications

Most modifications on the sugar group occur at the 2′-OH of the RNA sugar ring, which provides a convenient chemically reactive site Manoharan, M. 2004. RNA interference and chemically modified small interfering RNAs. Curr Opin Chem Biol 8:570-9; Zhang, H. Y., Du, Q., Wahlestedt, C., Liang, Z. 2006. RNA Interference with chemically modified siRNA. Curr Top Med Chem 6:893-900). The 2′-F and 2′-OME (0.7 and 8) are common and both increase stability, the 2′-OME modification does not reduce activity as long as it is restricted to less than 4 nucleotides per strand (Holen, T., Amarzguioui, M., Babaie, E., Prydz, H.2003. Similar behaviour of single-strand and double-strand siRNAs suggests they act through a common RNAi pathway. Nucleic Acids Res 31:2401-7). The 2′-O-MOE (0.9) is most effective in siRNA when modified bases are restricted to the middle region of the molecule (Prakash, T. P., Allerson, C. R., Dande, P., Vickers, T. A., Sioufi, N., Jarres, R., Baker, B. F., Swayze, E. E., Griffey, R. H., Bhat, B. 2005. Positional effect of chemical modifications on short interference RNA activity in mammalian cells. J Med Chem 48:4247-53). Other modifications found to stabilize siRNA without loss of activity are shown in 0.10-14.

Modified oligonucleotides may also contain one or more substituted sugar moieties. For example, the invention includes oligonucleotides that comprise one of the following at the 2′ position: OH; F; O—, S—, or N-alkyl, O-alkyl-O-alkyl, O—, S—, or N-alkenyl, or O—, S— or N-alkynyl, wherein the alkyl, alkenyl and alkynyl may be substituted or unsubstituted C₁ to C₁₀ alkyl or C2 to C10 alkenyl and alkynyl. Particularly preferred are O[(CH₂)ₙO]ₘCH₃, O(CH₂)ₙOCH₃, O(CH₂)₂ON(CH₃)₂, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. One modification includes 2′-methoxyethoxy(2′-O—CH₂CH₂OCH₃, also known as 2′-O-(2-methoxyethyl) or 2′-MOE) (Martin et al., Helv. Chim. Acta 1995, 78, 486-504), i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy(2′-DMAEOE).

Additional modifications include 2′-methoxy(2′-O—CH₃), 2′-aminopropoxy(2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugars structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920.

In other oligonucleotide mimetics, both the sugar and the internucleoside linkage, i.e., the backbone, of the nucleotide units are replaced with novel groups, although the base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262. Further teaching of PNA compounds can be found in Nielsen et al. (Science, 1991, 254, 1497-1500).

Particular embodiments of the invention are oligonucleotides with phosphorothioate backbones and oligonucleosides with heteroatom backbones, and in particular —CH₂—NH—O—CH₂—, —CH₂—N(CH₃)—O—CH₂— (referred to as a methylene (methylimino) or MMI backbone) —CH₂—O—N(CH₃)—CH₂—N(CH₃)—N(CH₃)—CH₂— and —O—N(CH₃)—CH₂—CH₂— (wherein the native phosphodiester backbone is represented as —O—P—O—CH₂—) of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

### d. Chimeric Oligonucleotides

It is not necessary for all positions in a given compound to be uniformly modified, and in fact more than one of the aforementioned modifications may be incorporated in a single compound or even at a single nucleoside within an oligonucleotide. Certain preferred oligonucleotides of this invention are chimeric oligonucleotides. “Chimeric oligonucleotides” or “chimeras,” in the context of this invention, are oligonucleotides that contain two or more chemically distinct regions, each made up of at least one nucleotide. These oligonucleotides typically contain at least one region of modified nucleotides that confers one or more beneficial properties (such as, e.g., increased nuclease resistance, increased uptake into cells, increased binding affinity for the RNA target). In one embodiment, a chimeric oligonucleotide comprises at least one region modified to increase target binding affinity. Affinity of an oligonucleotide for its target is routinely determined by measuring the Tm of an oligonucleotide/target pair, which is the temperature at which the oligonucleotide and target dissociate; dissociation is detected spectrophotometrically. The higher the Tm, the greater the affinity of the oligonucleotide for the target. In one embodiment, the region of the oligonucleotide which is modified to increase target mRNA binding affinity comprises at least one nucleotide modified at the 2′ position of the sugar, most preferably a 2′-O-alkyl, 2′-O-alkyl-O-alkyl or 2′-fluoro-modified nucleotide. Such modifications are routinely incorporated into oligonucleotides and these oligonucleotides have been shown to have a higher Tm (i.e., higher target binding affinity) than 2′-deoxyribo-oligonucleotides against a given target. The effect of such increased affinity is to greatly enhance oligonucleotide inhibition of target gene expression.

In another embodiment, a chimeric oligonucletoide comprises a region that acts as a substrate for RNAse H. Of course, it is understood that oligonucleotides may include any combination of the various modifications described herein

Another modification of the oligonucleotides of the invention involves chemically linking to the oligonucleotide one or more moieties or conjugates which enhance the activity, cellular distribution or cellular uptake of the oligonucleotide. Such conjugates and methods of preparing the same are known in the art.

The oligonucleotides used in accordance with this invention may be conveniently and routinely made through the well-known technique of solid phase synthesis. Equipment for such synthesis is sold by several vendors including Applied Biosystems. Any other means for such synthesis may also be employed; the actual synthesis of the oligonucleotides is well within the talents of the routineer. It is also well known to use similar techniques to prepare other oligonucleotides such as the phosphorothioates and alkylated derivatives.

Pharmaceutical Compositions

The lipophilic conjugated oliogonucleotides of present invention may be formulated as a pharmaceutical composition, e.g., which further comprises a pharmaceutically acceptable diluent, excipient, or carrier, such as physiological saline or phosphate buffer, selected in accordance with the route of administration and standard pharmaceutical practice.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids. Particularly perfered are formulations that target the liver when treating hepatic disorders such as hepatic carcinoma.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Emulsions

The compositions of the present invention may be prepared and formulated as emulsions. Emulsions are typically heterogenous systems of one liquid dispersed in another in the form of droplets usually exceeding 0.1 .mu.m in diameter (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions may be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions may contain additional components in addition to the dispersed phases, and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of dsRNAs and nucleic acids are formulated as microemulsions. A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or dsRNAs. Microemulsions have also been effective in the transdermal delivery of active components in both cosmetic and pharmaceutical applications. It is expected that the microemulsion compositions and formulations of the present invention will facilitate the increased systemic absorption of dsRNAs and nucleic acids from the gastrointestinal tract, as well as improve the local cellular uptake of dsRNAs and nucleic acids.

Microemulsions of the present invention may also contain additional components and additives such as sorbitan monostearate (Grill 3), Labrasol, and penetration enhancers to improve the properties of the formulation and to enhance the absorption of the dsRNAs and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

Liposomes

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Desirable considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Other Components

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

Method of Use

The conjugated oligonucleotides of the present invention may be used to deliver a therapeutic agent to a cell, in vitro or in vivo. While the following description of various methods of using the conjugated oligonucleotides and related pharmaceutical compositions of the present invention are exemplified by description, it is understood that these methods and compositions may be readily adapted for the delivery of any therapeutic agent for the treatment of any disease or disorder that would benefit from such treatment.

In certain embodiments, the present invention provides methods for introducing a nucleic acid into a cell in the epithelial tissues. Preferred nucleic acids for introduction into cells are siRNA, microRNA, immune-stimulating oligonucleotides, plasmids, antisense and ribozymes. These methods may be carried out by contacting the compositions of the present invention with the cells for a period of time sufficient for intracellular delivery to occur.

The methods of the present invention may be practiced in a variety of hosts. Preferred hosts include mammalian species, such as humans, non-human primates, dogs, cats, cattle, horses, sheep, and the like.

In one embodiment, the present invention provides a method of modulating the expression of a target polynucleotide or polypeptide. These methods generally comprise contacting a cell with a lipophilic conjugated oligonucleotides of the present invention that is associated with a nucleic acid capable of modulating the expression of a target polynucleotide or polypeptide. As used herein, the term “modulating” refers to altering the expression of a target polynucleotide or polypeptide. In different embodiments, modulating can mean increasing or enhancing, or it can mean decreasing or reducing. Methods of measuring the level of expression of a target polynucleotide or polypeptide are known and available in the arts and include, e.g., methods employing reverse transcription-polymerase chain reaction (RT-PCR) and immunohistochemical techniques. In particular embodiments, the level of expression of a target polynucleotide or polypeptide is increased or reduced by at least 10%, 20%, 30%, 40%, 50%, or greater than 50% as compared to an appropriate control value.

For example, if increased expression of a polypeptide is desired, the nucleic acid may be an expression vector that includes a polynucleotide that encodes the desired polypeptide. On the other hand, if reduced expression of a polynucleotide or polypeptide is desired, then the nucleic acid may be, e.g., an antisense oligonucleotide, siRNA, or microRNA that comprises a polynucleotide sequence that specifically hybridizes to a polnucleotide that encodes the target polypeptide, thereby disrupting expression of the target polynucleotide or polypeptide. Alternatively, the nucleic acid may be a plasmid that expresses such an antisense oligonucletoide, siRNA, or microRNA.

In particular embodiments, the therapeutic agent is selected from an siRNA, a microRNA, an antisense oligonucleotide, and a plasmid capable of expressing an siRNA, a microRNA, or an antisense oligonucleotide, and wherein the siRNA, microRNA, or antisense RNA comprises a polynucleotide that specifically binds to a polynucleotide that encodes the polypeptide, or a complement thereof, such that the expression of the polypeptide is reduced.

In related embodiments, the present invention provides a method of treating a disease or disorder characterized by overexpression of a polypeptide in a subject, comprising providing to the subject a pharmaceutical composition of the present invention, wherein the therapeutic agent is selected from an siRNA, a microRNA, an antisense oligonucleotide, and a plasmid capable of expressing an siRNA, a microRNA, or an antisense oligonucleotide, and wherein the siRNA, microRNA, or antisense RNA comprises a polynucleotide that specifically binds to a polynucleotide that encodes the polypeptide, or a complement thereof.

A variety of tumor antigens, infections agent antigens, and antigens associated with other disease are well known in the art and examples of these are described in references cited herein. Examples of antigens suitable for use in the present invention include, but are not limited to, polypeptide antigens and DNA antigens. Specific examples of antigens are Hepatitis A, Hepatitis B, small pox, polio, anthrax, influenza, typhus, tetanus, measles, rotavirus, diphtheria, pertussis, tuberculosis, and rubella antigens. In one embodiment, the antigen is a Hepatitis B recombinant antigen. In other aspects, the antigen is a Hepatitis A recombinant antigen. In another aspect, the antigen is a tumor antigen. Examples of such tumor-associated antigens are MUC-1, EBV antigen and antigens associated with Burkitt's lymphoma. In a further aspect, the antigen is a tyrosinase-related protein tumor antigen recombinant antigen. Those of skill in the art will know of other antigens suitable for use in the present invention.

Tumor-associated antigens suitable for use in the subject invention include both mutated and non-mutated molecules that may be indicative of single tumor type, shared among several types of tumors, and/or exclusively expressed or overexpressed in tumor cells in comparison with normal cells. In addition to proteins and glycoproteins, tumor-specific patterns of expression of carbohydrates, gangliosides, glycolipids and mucins have also been documented. Exemplary tumor-associated antigens for use in the subject cancer vaccines include protein products of oncogenes, tumor suppressor genes and other genes with mutations or rearrangements unique to tumor cells, reactivated embryonic gene products, oncofetal antigens, tissue-specific (but not tumor-specific) differentiation antigens, growth factor receptors, cell surface carbohydrate residues, foreign viral proteins and a number of other self proteins.

Specific embodiments of tumor-associated antigens include, e.g., mutated antigens such as the protein products of the Ras p21 protooncogenes, tumor suppressor p53 and BCR-abl oncogenes, as well as CDK4, MUM1, Caspase 8, and Beta catenin; overexpressed antigens such as galectin 4, galectin 9, carbonic anhydrase, Aldolase A, PRAME, Her2/neu, ErbB-2 and KSA, oncofetal antigens such as alpha fetoprotein (AFP), human chorionic gonadotropin (hCG); self antigens such as carcinoembryonic antigen (CEA) and melanocyte differentiation antigens such as Mart 1/Melan A, gp100, gp75, Tyrosinase, TRP1 and TRP2; prostate associated antigens such as PSA, PAP, PSMA, PSM-P1 and PSM-P2; reactivated embryonic gene products such as MAGE 1, MAGE 3, MAGE 4, GAGE 1, GAGE 2, BAGE, RAGE, and other cancer testis antigens such as NY-ESO₁, SSX2 and SCP1; mucins such as Muc-1 and Muc-2; gangliosides such as GM2, GD2 and GD3, neutral glycolipids and glycoproteins such as Lewis (y) and globo-H; and glycoproteins such as Tn, Thompson-Freidenreich antigen (TF) and sTn. Also included as tumor-associated antigens herein are whole cell and tumor cell lysates as well as immunogenic portions thereof, as well as immunoglobulin idiotypes expressed on monoclonal proliferations of B lymphocytes for use against B cell lymphomas.

Pathogens include, but are not limited to, infectious agents, e.g., viruses, that infect mammals, and more particularly humans. Examples of infectious virus include, but are not limited to: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronoviridae (e.g., coronaviruses); Rhabdoviradae (e.g., vesicular stomatitis viruses; rabies viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae herpes simplex virus (HSV) 1 and 2, varicella zoster virus, cytomegalovirus (CMV), herpes virus; Poxyiridae (variola viruses, vaccinia viruses, pox viruses); and Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Also, gram negative and gram positive bacteria serve as antigens in vertebrate animals. Such gram positive bacteria include, but are not limited to Pasteurella species, Staphylococci species, and Streptococcus species. Gram negative bacteria include, but are not limited to, Escherichia coli, Pseudomonas species, and Salmonella species. Specific examples of infectious bacteria include but are not limited to: Helicobacter pyloris, Borelia burgdorferi, Legionella pneumophilia, Mycobacteria sps (e.g., M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus infuenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Additional examples of pathogens include, but are not limited to, infectious fungi that infect mammals, and more particularly humans. Examples of infectious fingi include, but are not limited to: Cryptococcus neoformans, Histoplasma capsulatum, Coccidioides immitis, Blastomyces dermatitidis, Chlamydia trachomatis, Candida albicans. Examples of infectious parasites include Plasmodium such as Plasmodium falciparum, Plasmodium malariae, Plasmodium ovale, and Plasmodium vivax. Other infectious organisms (i.e., protists) include Toxoplasma gondii.

Example of Synthesis of Conjugated siRNAs

siRNAs conjugated to cholesterol were prepared according to scheme 1. It is understood that other conjugates can be linked to the oligonucleotides via a similar method known to one of ordinary skill in the art, such methods can be found in US publication nos. 2005/0107325, 2005/0164235, 2005/0256069 and 2008/0108801, which are hereby incorporated by reference in their entirety.

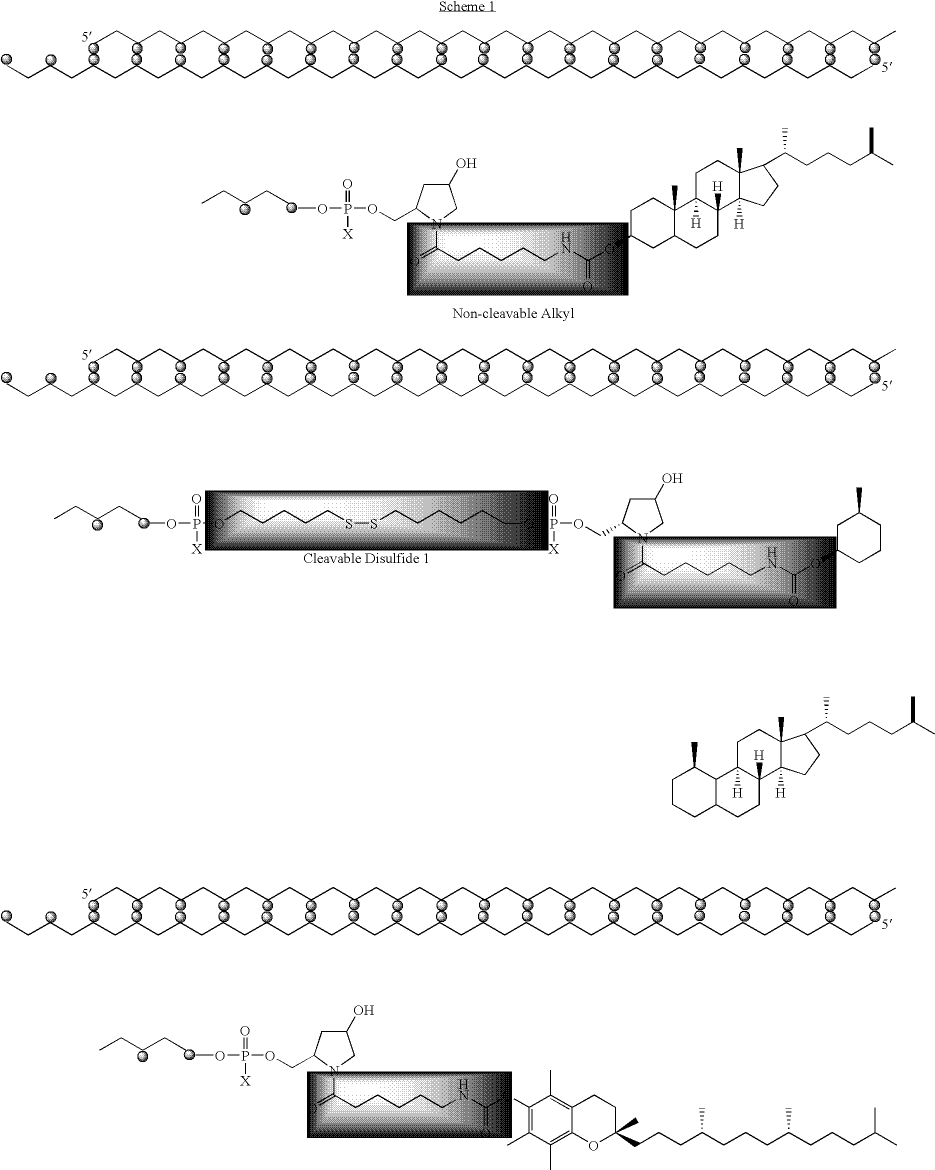

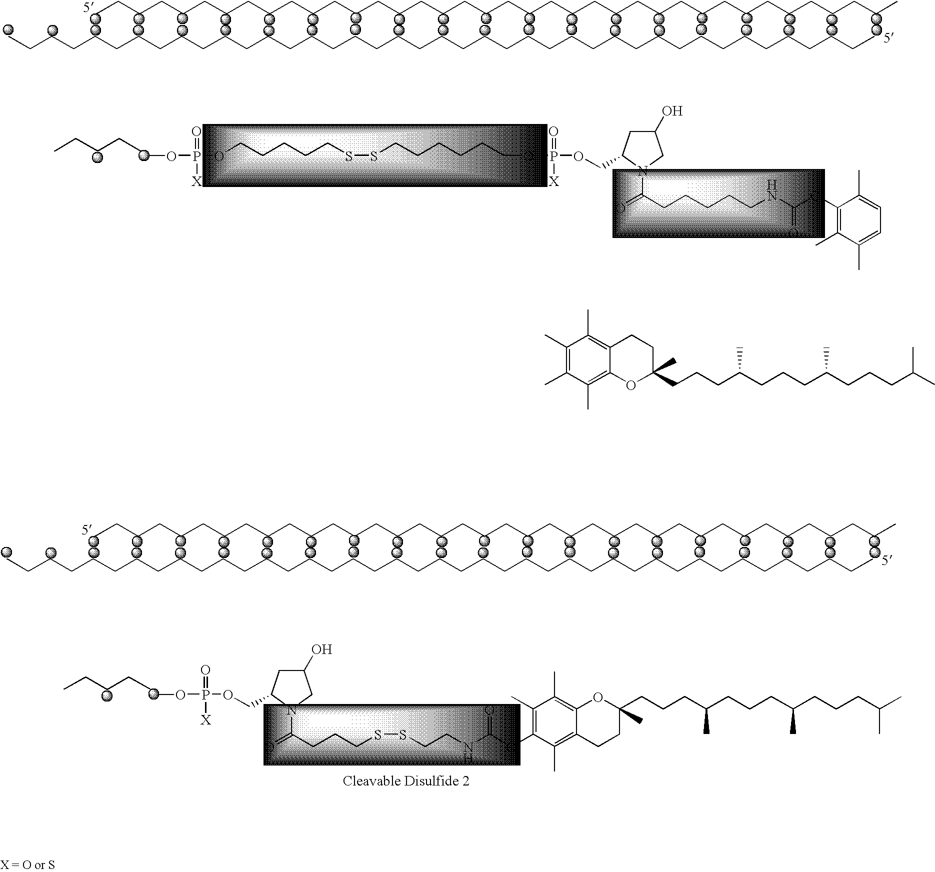

### EXAMPLES

### Example 1

### Evaluation of siRNA Distribution In Vivo without Ethanol

### Protocol:

### Day −5 to −7:

### Cycling to Diestrus

6-8 week old female C57/BL6 mice receive 3 mg medroxyprogesterone (200 ul 15 mg/ml subcutaneous)

### Day 0:

### Mucous Removal Prewash and Dosing

Mice Anesthetized and dosed with 30 ul 10 mg/ml Acetylcysteine in 1×PBS
Acetylcysteine removed
30 ul of 2×PBS administered
2×PBS removed
Vaginal Canal cleared with cotton swab
20 ul of siRNA administered

### Day 0: (˜2 and ˜4-5 Hours Post Formulation Dose)

Canal washed with 1×PBS prior to takedown to remove any residual formulation
Intact vaginal canal and cervix harvested and processed for frozen tissue sections
   [symbol] 7 micron sections taken throughout tissue—10-12 sections per sample˜30-40 sections apart
Sections DAPI stained for microscopy
   Fluorescent signal (e.g. Cy3 or Alexa488) captured such that image is representative of what is seen under the microscope. A second image is captured in the opposite channel of that of the tag (e.g. green channel for Cy3 tagged siRNAs) at the same exposure (matched image) to confirm that signal seen in channel of tag is above that of the tissue autofluorescence. When comparing across formulations within the same experiment, the exposure time is held constant across samples for accurate comparison. Images are captured in this manner at 10×, 20×, and at higher magnification, as needed.

TABLE 1
Cy3-tagged ApoB siRNA Conjugates for
Distribution in Mouse Vaginal/Cervical Model

| Duplex | | | | ssRNA | |
|---|---|---|---|---|---|
| # | Target | Description | Strand | # | Sequence 5′-3′ |
| 18560 | ApoB | S unconj/AS 5′ Cy3 | S | 5296 | GGAAUCuuAuAuuuGAUCcAsA |
| | | | AS | 31849 | Q38uuGGAUcAAAuAuAAGAuUCcscsU |
| 18117 | ApoB | S 3′Chol/AS 5′ Cy3 | S | 5474 | GGAAUCuuAuAuuuGAUCcAAs-Chol |
| | | | AS | 31849 | Q38uuGGAUcAAAuAuAAGAuUCcscsU |
| 18561 | ApoB | S 3′ C16-Chol/AS 5′ Cy3 | S | 30602 | GGAAUCuuAuAuuuGAUCcAAs-C16-Chol |
| | | | AS | 31849 | Q38uuGGAUcAAAuAuAAGAuUCcscsU |
| 18562 | ApoB | S 3′ PEG4-Chol/ | S | 30669 | GGAAUCuuAuAuuuGAUCcAAsL94 |
| | | AS 5′ Cy3 | AS | 31849 | Q38uuGGAUcAAAuAuAAGAuUCcscsU |
| 18563 | ApoB | S 3′ s-s-Chol/AS 5′ Cy3 | S | 30667 | GGAAUCuuAuAuuuGAUCcAAsL92 |
| | | | AS | 31849 | Q38uuGGAUcAAAuAuAAGAuUCcscsU |
| 18564 | ApoB | S 3′ C6-Docosanoyl/ | S | 30604 | GGAAUCuuAuAuuuGAUCcAAs-C22 |
| | | AS 5′ Cy3 | AS | 31849 | Q38uuGGAUcAAAuAuAAGAuUCcscsU |
| 18565 | ApoB | S 3′ LCO/AS 5′ Cy3 | S | 30859 | GGAAUCuuAuAuuuGAUCcAAsL98 |
| | | | AS | 31849 | Q38uuGGAUcAAAuAuAAGAuUCcscsU |
| 18566 | ApoB | S 3′ UDC/AS 5′ Cy3 | S | 30985 | GGAAUCuuAuAuuuGAUCcAAsL14 (Hyp-C6-NH2-UDC) |
| | | | AS | 31849 | Q38uuGGAUcAAAuAuAAGAuUCcscsU |

TABLE 2
K14 siRNA

| Duplex # | Target | Description | Strand | ssRNA # | Sequence 5′-3′ |
|---|---|---|---|---|---|
| 18365 | Keratin14 | 2′Fluoro modified Cy3 | S | 22340 | GUfAUfUfGUfCfCfCfUfAUfAGGCfUfUfdTsdT |
| | | tagged siRNA | AS | 31913 | Q38AAGCfCfUfAUfAGGGACfAAUfACfdTsdT |
| 18366 | Keratin14 | 2′Fluoro modified Cy3 | S | 30098 | GUfAUfUfGUfCfCfCfUfAUfAGGCfUfUfd |
| | | tagged Chol-siRNA | | | TsdTL10 |
| | | | AS | 31913 | Q38AAGCfCfUfAUfAGGGACfAAUfACfdTsdT |

TABLE 3
Abbrevations

| Abbreviation | Nucleotide(s) |
|---|---|
| A | adenosine-3′-phosphate |
| C | cytidine-3′-phosphate |
| G | guanosine-3′-phosphate |
| T | 5-methyluridine-3′-phosphate |
| U | uridine-3′-phosphate |
| N | any nucleotide (G, A, C, or T) |
| a | 2′-O-methyladenosine-3′-phosphate |
| c | 2′-O-methylcytidine-3′-phosphate |
| g | 2′-O-methylguanosine-3′-phosphate |
| u | 2′-O-methyluridine-3′-phosphate |
| dT | 2′-deoxythymidine-3′-phosphate |
| s | Phosphorothioate |
| Uf | 2′-Fluorouridine |
| Cf | 2′-Fluorocytodine |
| Q38 | Quasar 570 phosphate (BNS-5063, Biosearch Tech) |
| L92 | N-(cholesterylcarboxamidoethyl-dithio-butyryl)-4- |
| | hydroxyprolinol (Hyp-S-S-Chol) |
| L94 | N-(cholesterylcarboxamido-PEG4-proprionyl)-4- |
| | hydroxyprolinol (Hyp-PEG4-Chol) |
| L98 | N-(lithocholicoleylcarboxamidocaproyl)-4-hydroxyprolinol |
| | (Hyp-C6-LCO, amide) |
| L14 | N-(ursodeoxycholylcarboxamidoethyl-butyryl)-4- |
| | hydroxyprolinol (Hyp-ursodeoxycholic acid) |
| L10 | N-(cholesterylcarboxamidocaproyl)-4-hydroxyprolinol |
| | (Hyp-C6-Chol) |
| L89 | N-(cholesterylcarboxamidohexadecanoyl)-4- |
| | hydroxyprolinol (Hyp-C16-Chol) |
| L58 | N-(docosanylcarboxamidocaproyl)-4-hydroxyprolinol |
| | (Hyp-C6-C22) |

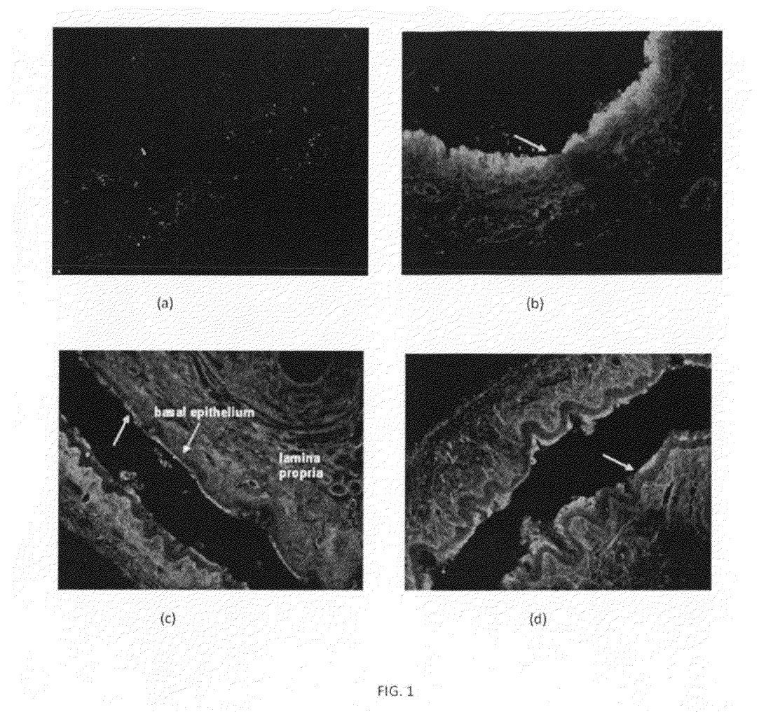

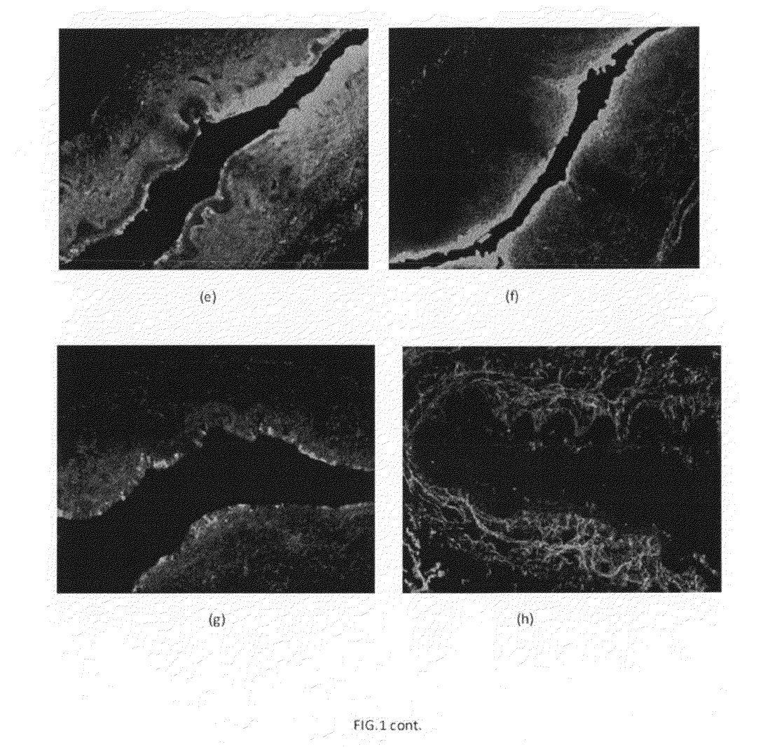

As shown in FIG. 1, the distribution of the lipophilic conjugates is deep into the vaginal epithelium. Lithocholicoleoyl (c), disulfide-cholesterol (d), C16-cholesterol (e) and C22 (g) conjugated siRNAs penetrate deep into the vaginal epithelium and appear to penetrate deeper than the cholesterol conjugated-siRNA (b). PEG4-cholesterol-siRNA (f) behaves similarly to cholesterol-siRNA. Deoxycholanic acid conjugated siRNA (h) does not appear to permeate the vaginal epithelium to the same degree or with the same pattern as the other conjugates. Cy3 alone (a, i.e. no conjugate) is not sufficient for the observed tissue coverage and permeation as compared to the lipophilic conjugates.

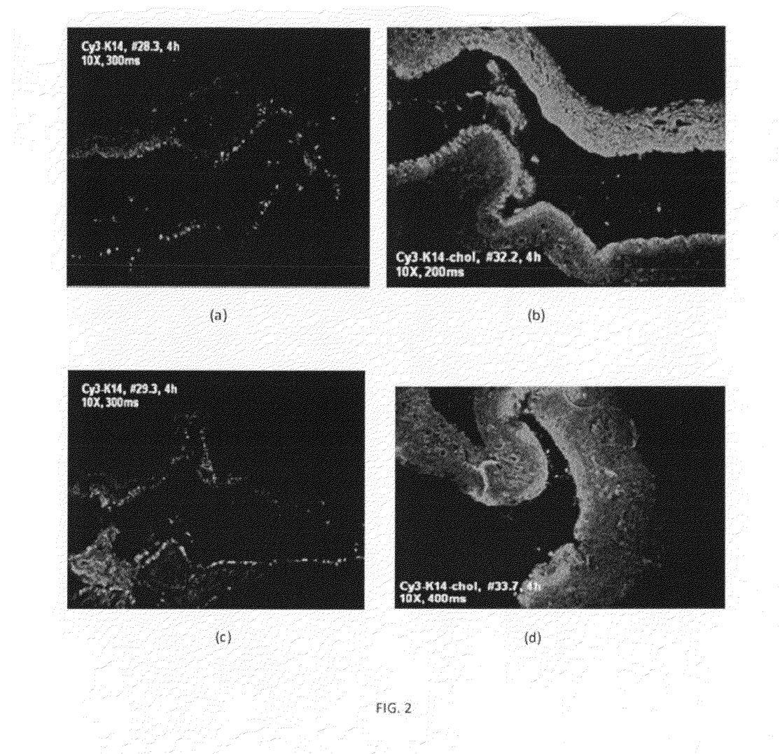

The desireability of a lipophilic conjugate is further illustrated in FIG. 2. Cholesterol conjugate contributes to the permeation of K14 siRNA into the vaginal epithelium. Cholestrerol conjugated K14 demonstrates good permeation whereas no permeation was observed in the absence of cholesterol conjugate (compare FIG. 2b,d chol-conjugated siRNA to FIG. 2a,c unconjugated siRNA).

### Example 2

### Evaluation of siRNA Distribution In Vivo in the Presence of Ethanol

This study was done similarly to the protocol of Example 1, but the siRNA was formulated with ethanol. Ethanol was added to siRNA prepared in 1×PBS just prior to dosing to the desired dosing concentration of siRNA and % by volume of Ethanol and the solution was mixed thouroughly by vortexing. The final PBS concentration of dosing solution was typically between 0.9-0.995× depending on the desired Ethanol concentration.

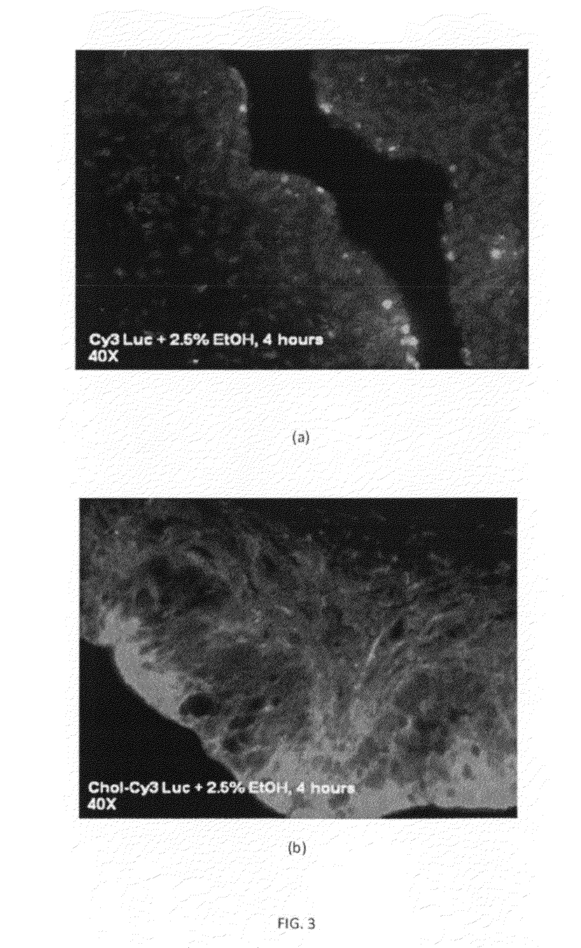

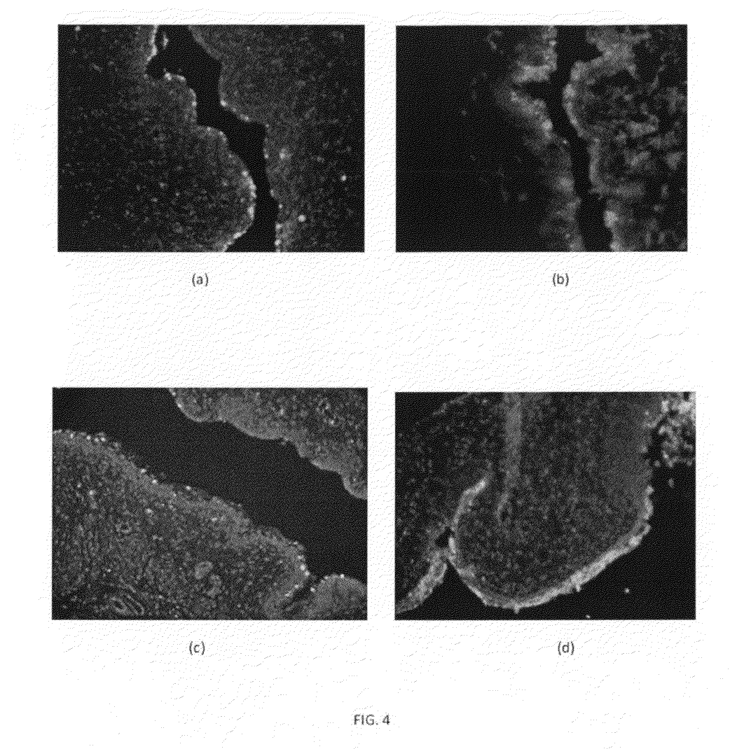

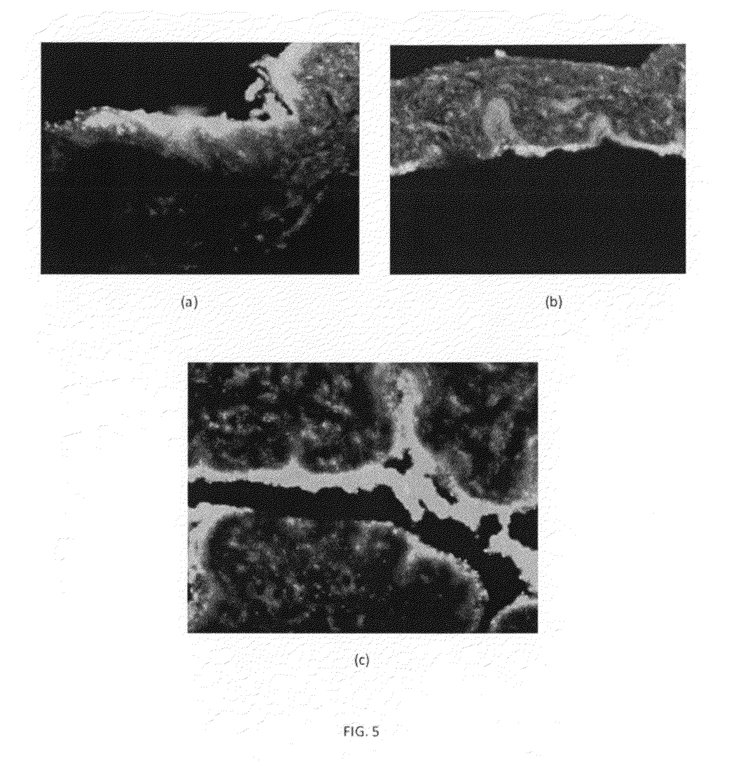

As shown in FIG. 3, no significant permeation is seen for Cy3 tagged Luc siRNA at 2.5% EtOH (FIG. 3a), while significant permeation is seen throughout the basal epithelial and into the lamina propia for Cy3-tagged Luc-Chol siRNA with 2.5% EtOH. The same observation was found with a Luc-Chol siRNA tagged with a different fluorophore (Alexa488) indicating that the fluorophore tag did not influence the distribution pattern observed for the cholesterol conjugated siRNA (FIG. 4).

TABLE 4
Fluorophore-tagged Luc siRNAs for Distribution in Mouse Vaginal/Cervical Model

| Duplex # | Target | Description | Strand | ssRNA # | Sequence 5′-3′ |
|---|---|---|---|---|---|
| 3345 | Luciferase | Cy3-Luc | S | 3372 | cuuAcGcuGAGuAcuucGAdTsdT |
| | | | AS | 30186 | Q38ucGAAGuAcucAGcGuAAGdTsdT |
| 3356 | Luciferase | Alexa488-Luc | S | 3372 | cuuAcGcuGAGuAcuucGAdTsdT |
| | | | AS | 30187 | Alexa488-ucGAAGuAcucAGcGuAAGdTsdT |
| 3570 | Luciferase | 3′Chol-S/ | S | 3373 | cuuAcGcuGAGuAcuucGAdTsdTsL10 |
| | | 5′Cy3-AS | AS | 30186 | Q38ucGAAGuAcucAGcGuAAGdTsdT |
| 3571 | Luciferase | 3′Chol-S/ | S | 3373 | cuuAcGcuGAGuAcuucGAdTsdTsL10 |
| | | 5′Alexa488-AS | AS | 30187 | Alexa488-ucGAAGuAcucAGcGuAAGdTsdT |

### Example 3

### Evaluation of siRNA Distribution In Vivo at Various Ethanol Concentrations

### Protocol:

Day −4-6:
C57/BL6 mice received progesterone 3 mg

Day 0:
Mice Anesthetized and dosed with 30 ul 110 mg/ml acetyl cysteine in 1×PBS
Mucous/liquid purged
30 ul of 2×PBS administered
Mucous/liquid purged
Vaginal canal cleared with cotton swab
20 ul of formulation administered

Day 0: (1 or 4-5 hours post formulation dose)
Canal washed with 1×PBS prior to takedown to remove any residual formulation
Intact vaginal canal and cervix harvested and processed for frozen tissue sections 7 uM sections taken throughout tissue—10-12 sections per sample ˜30-40 sections apart
Sections DAPI stained for microscopy

TABLE 5
Experimental Groups for Evaluation of Cholesterol Conjugated siRNA at Various Ethanol Concentrations

| | | | | | Time Point | | Formulation | ug |
|---|---|---|---|---|---|---|---|---|
| Group | Formulation | siRNA | Duplex# | N | 4 h | 18 h | Concentration (siRNA) | siRNA/dose |
| 1 | Alexa Luc-chol + 0.5% EtOH | Alexa488 tagged | 3571 | 4 | 3 | 3 | 2.5 mg/ml | 50 |
| 2 | Alexa Luc-Chol + 1% EtOH | Luc-Cholesterol | | 4 | 2 | 2 | | |
| 3 | Alexa Luc-Chol + 2.5% EtOH | | | 6 | 2 | 2 | | |

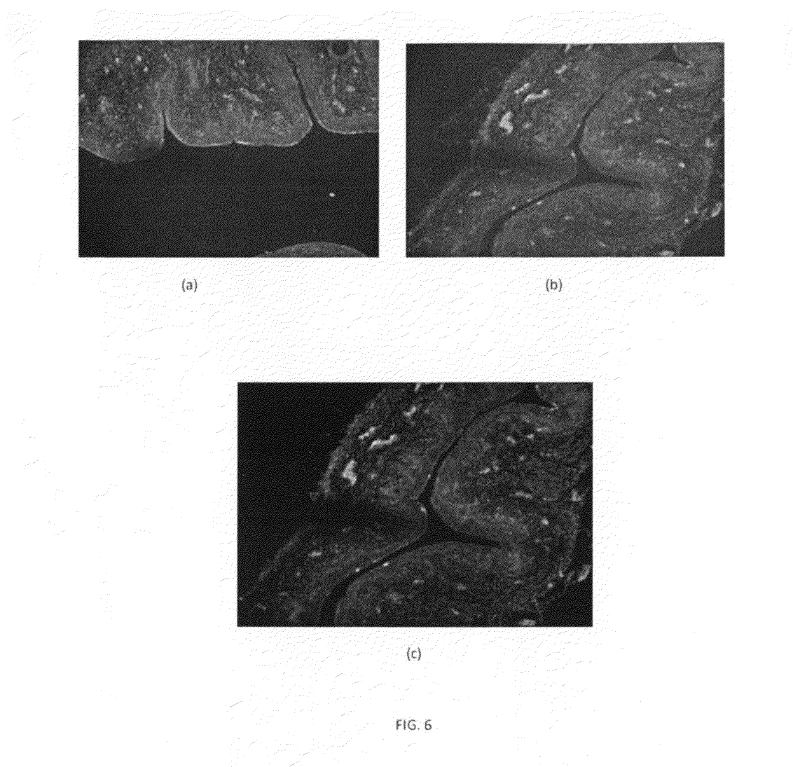

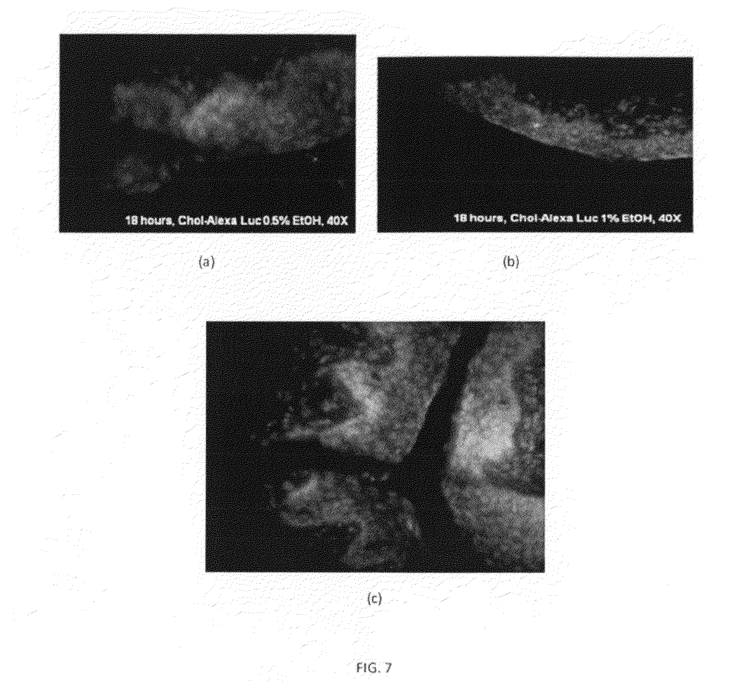

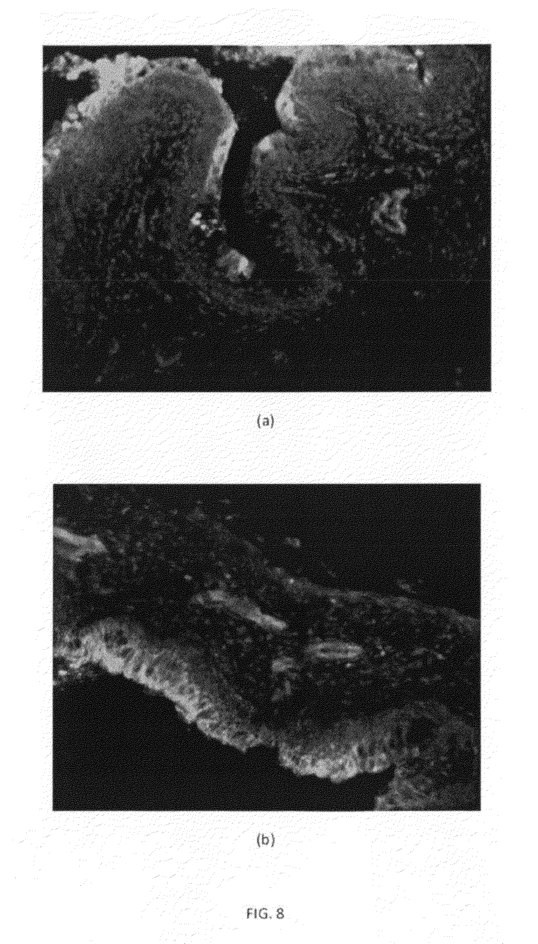

As illustrated in FIG. 5, permeation is seen throughout all layers of the basal epithelia at 0.5% and 1% EtOH concentrations. At 2.5% EtOH, distribution pattern appears to be the most prominent in coverage and intensity. Signal dissipated at 18 hours for 0.5%, 1% and 2.5% EtOH concentrations (see FIG. 6), however the signal that can be parsed out can still be seen throughout the basal epithelia layer (see FIG. 7). At 17.5 hours with 10% EtOH, signal is still present (see FIG. 8). Higher ethanol concentration may be used to maintain long term exposure. The dissipation of signal at the late time point may suggest the need for multiple dosing when using lower EtOH concentrations.

### Example 4

### Evaluation of siRNA Efficacy In Vivo 1^{st} Trial

### Protocol:

### Day −5 to −7:

### Cycling to Diestrus

6-8 week old female C57BL/6 or Balb/C mice (Charles River Lab) receive medroxyprogesterone 3 mg (200 ul 15 mg/ml subcutaneous)

### Day 0 and Day 1:

### Mucous Removal Prewash and Dosing

Mice Anesthetized and dosed with 30 ul 10 mg/ml Acetylcysteine in 1×PBS
Acetylcysteine removed
30 ul of 2×PBS administered
2×PBS removed
Vaginal Canal cleared with cotton swab
10 ul 5 mg/ml chol-siRNA in 0.95×PBS, 5% EtOH is administered
A Cytobrush® Plus Gentle Touch Cell Collector (Cooper Surgical) is inserted and twisted 10 times to disrupt epithelium (cytobrush method adapted from Roberts, J. N. et al. Genital transmission of HPV in a mouse model is potentiated by nonoxynol-9 and inhibited by carrageenan. Nat. Medicine 13: 857-861 (2007))
An additional 10 ul 5 mg/ml chol-siRNA in 0.95×PBS, 5% EtOH is administered

### Day 2:

### Take Down

Whole vaginal canal and cervix are harvested separately and flash frozen
Tissues are sonicated in 1 ml Epicentre tissue and cell lysis buffer with 300 μg/ml ProteinaseK
Incubated at 65° C. for 45 minutes at 900 rpm
mRNA levels evaluated by QuantiGene™ 1.0 bDNA

TABLE 6
Experimental Groups for Evaluation of in vivo Efficacy of Cholesterol Conjugated
siRNA with Cytobrush ® Plus Gentle Touch Cell Collector
In vivo efficacy with E6AP-Chol and K14-Chol siRNAs + 5% Ethanol with Cytobrush Mechanical Disruption

| | | | | | | Formulation | ug |
|---|---|---|---|---|---|---|---|
| Group | Formulation | siRNA | Duplex # | N | Dosing | Concentration (siRNA) | siRNA/dose |
| 1 | E6AP-Chol 5% EtOH | E6AP-Chol | 8885 | 10 | once/day 2 | 5 mg/ml | 100 |
| 2 | K14-CHol 5% EtOH | K14-CHol | 3175 | 10 | days, 24 h | | |
| 3 | 1X PBS + 5% EtOH | | | 5 | TD | — | — |
| 4 | 1X PBS | — | — | 5 | | | |
| | | | Total = | 30 | | | |

Acetyl-cysteine/hypertonic pre-treatment followed by dosing in isotonic conditions at neutral pH
C57Bl/6 mice received progesterone treatment on Apr. 11, 2008, experiment begins Apr. 16, 2008
mice were anesthetized and dosed with 30 ul 10 mg/ml acetyl-cysteine in 1XPBS
liquid/mucous is removed from vaginal canal and 30 ul of 2XPBS (548mOsmole) is adminstered
liquid/mucous is removed from vaginal canal and residual fluid removed with swab
10 ul formulation administered
Cytobrush inserted and twisted 10 times to disrupt cornified layer
10 ul formulation administered
Take Down
10 mice per group: vaginal canal and cervix frozen separately for bDNA analysis

TABLE 7
Cholesterol Conjugated siRNAs Evaluated in Efficacy Experiments

| Duplex # | Target | Description | Strand | ssRNA # | Sequence 5′-3′ |
|---|---|---|---|---|---|
| 3175 | Keratin14 | 2′Fluoro modified Chol-siRNA | S | 30098 | GUfAUfUfGUfCfCfCfUfAUfAGGCfUfUfdTdTL10 |
| | | | AS | 22346 | AAGCfCfUfAUfAGGGACfAAUfACfdTsdT |
| 8885 | E6AP | 2′OMe modified Chol-siRNA | S | 13204 | AcGAAuGAGuuuuGuGcuudTdTL10 |
| | | | AS | 13161 | AAGcAcAAAACUcAuUCGUdTsdT |

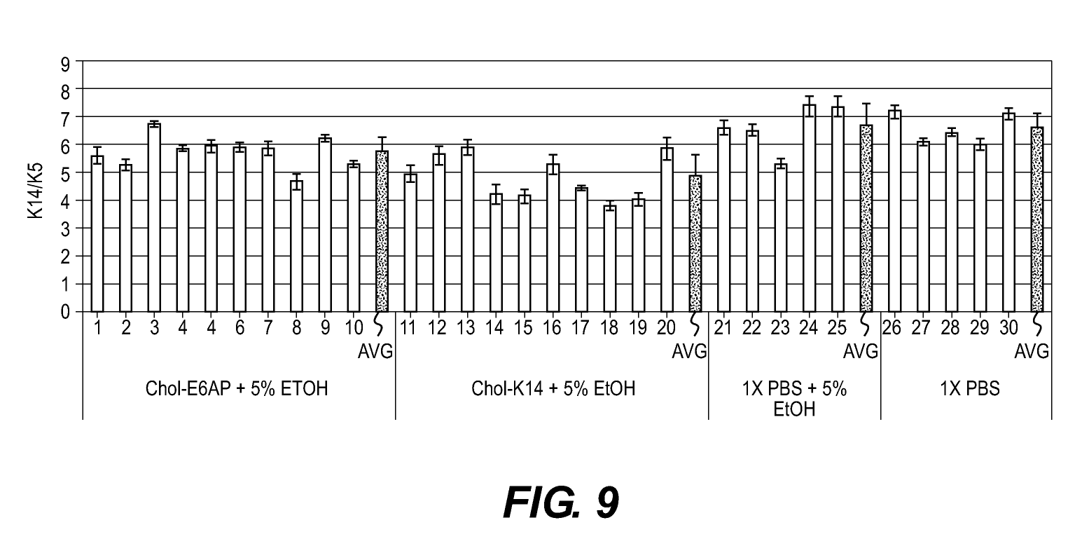

As can be seen from FIG. 9, 5 of 10 animals in the K14-chol siRNA treatment+5% EtOH group showed consistent reduction of K14 mRNA relative to the normalizing gene K5 in this first trial relative to both the E6AP-Chol siRNA+5% EtOH and PBS treatment groups.

### Example 5

### Evaluation of siRNA Efficacy In Vivo Follow-Up Trial

TABLE 8
In vivo efficacy with K14-Chol and E6AP-Chol siRNAs +/− 5% Ethanol
with Cytobrush ® Plus Gentle Touch Cell Collector

| | | | | | | Formulation | ug |
|---|---|---|---|---|---|---|---|
| Group | Formulation | siRNA | Duplex # | N | Dosing | Concentration (siRNA) | siRNA/dose |
| 1 | K14-Chol + 5% EtOH | K14-Chol | 3175 | 10 | once/day 2 | 5 mg/ml | 100 |
| 2 | E6AP-Chol + 5% EtOH | E6AP-Chol | 8885 | 10 | days, 24 hr | | |
| 3 | 1X PBS + 5% EtOH | — | — | 5 | TD | — | — |
| 4 | K14-Chol | K14-Chol | 3175 | 10 | | 5 mg/ml | 100 |
| 5 | E6AP-Chol | E6AP-Chol | 8885 | 10 | | | |
| 6 | 1X PBS | — | — | 5 | | — | — |
| | | | Total = | 50 | | | |

Acetyl-cysteine/hypertonic pre-treatment followed by dosing in isotonic conditions at neutral pH
C57Bl/6 mice DOB Jun. 4, 2008 received progesterone treatment on Jul. 25, 2008, experiment begins Jul. 30, 2008
mice were anesthetized and dosed with 30 ul 10 mg/ml acetyl-cysteine in 1XPBS
liquid/mucous is removed from vaginal canal and 30 ul of 2XPBS (548mOsmole) is adminstered
liquid/mucous is removed from vaginal canal and residual fluid removed with swab
10 ul formulation administered
Cytobrush inserted and twisted 10 times to disrupt epithelium
10 ul formulation administered
Take Down
10 mice per group: vaginal canal and cervix frozen separately for bDNA analysis

TABLE 9
Cholesterol Conjugated siRNAs Evaluated in Efficacy Experiments

| Duplex # | Target | Description | Strand | ssRNA # | Sequence 5′-3' |
|---|---|---|---|---|---|
| 3175 | Keratin14 | 2′Fluoro modified Chol-siRNA | S | 30098 | GUfAUfUfGUfCfCfCfUfAUfAGGCfUfUfdTdTL10 |
| | | | AS | 22346 | AAGCfCfUfAUfAGGGACfAAUfACfdTsdT |
| 8885 | E6AP | 2′OMe modified Chol-siRNA | S | 13204 | AcGAAuGAGuuuuGuGcuudTdTL10 |
| | | | AS | 13161 | AAGcAcAAAACUcAuUCGUdTsdT |

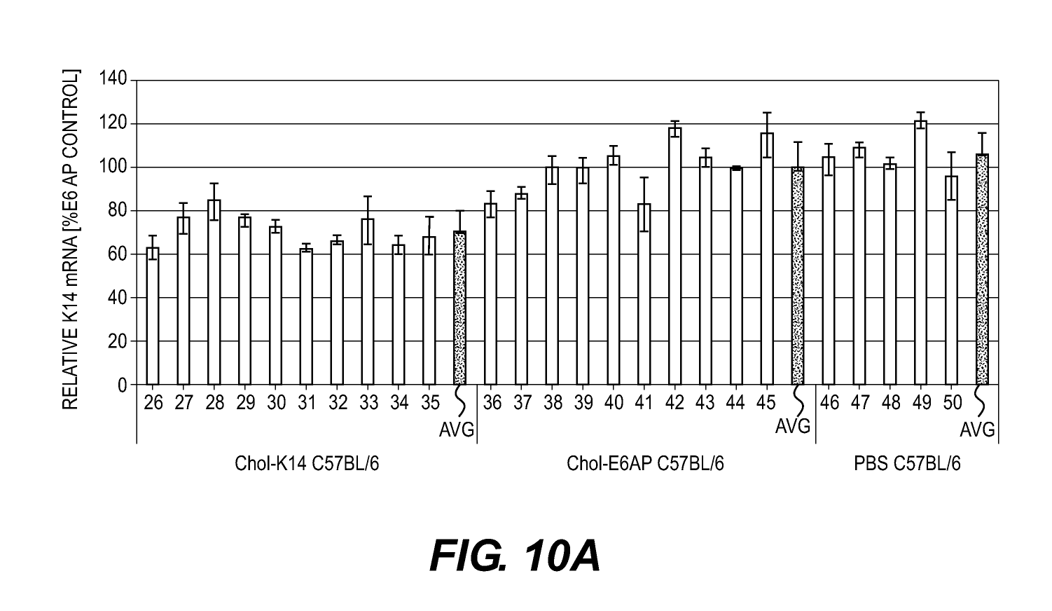

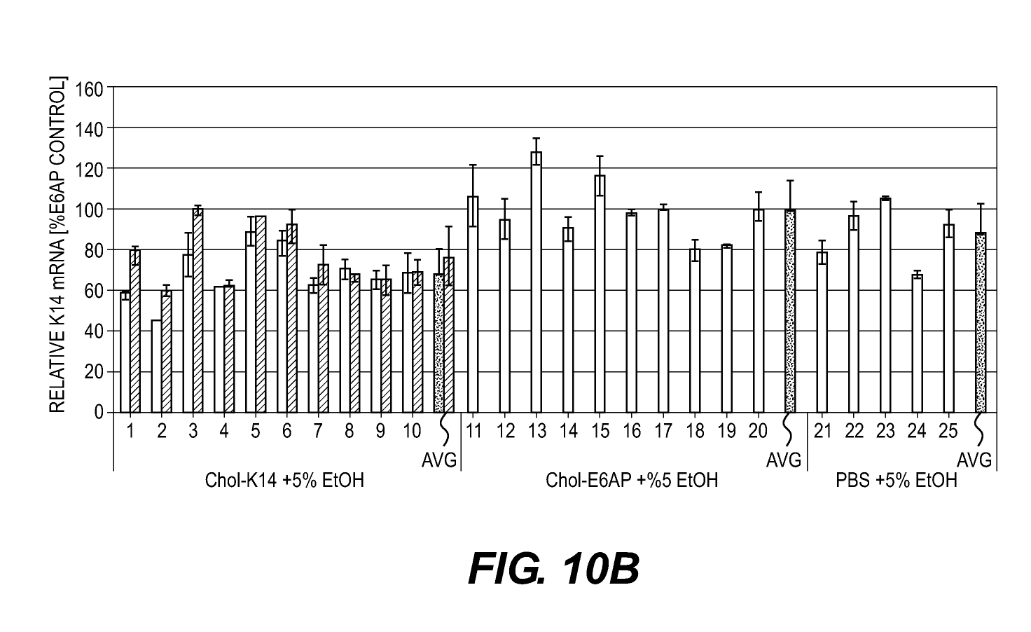

A follow-up experiment for K14-Chol siRNA with Cytobrush®+/−5% Ethanol was carried out. In the absence of ethanol, 29% K14 KD was observed with K14-Chol siRNA relative to vs E6AP-Chol siRNA treatement and 36% K14 KD was observed relative to PBS treatment (see FIG. 10a). In the presence of 5% EtOH, 38% K14 KD was found with K14-Chol siRNA relative to E6AP-Chol siRNA treatment and 44% K14 KD relative to PBS treatment (see FIG. 10b).

### Example 6

### Evaluation of siRNA Efficacy In Vivo of Chol-K14 siRNA w/Cytobrush®, 24 vs 48 Hour TD

TABLE 10
Chol Conjugated siRNA + 5% EtOH 24 vs. 48 hour takedown

| | | | | | | Formulation | ug |
|---|---|---|---|---|---|---|---|
| Group | Formulation | siRNA | Duplex # | N | Dosing | Concentration (siRNA) | siRNA/dose |
| 1 | K14-Chol + 5% EtOH | K14-Chol | 3175 | 10 | once/day 2 | 5 mg/ml | 100 |
| 2 | E6AP-Chol + 5% EtOH | E6AP-Chol | 8885 | 10 | days, 48 hr | | |
| 3 | 1X PBS + 5% EtOH | — | — | 5 | TD | — | — |
| 4 | K14-Chol + 5% EtOH | K14-Chol | 3175 | 10 | once/day 2 | 5 mg/ml | 100 |
| 5 | E6AP-Chol + 5% EtOH | E6AP-Chol | 8885 | 10 | days, 24 hr | | |
| 6 | 1X PBS + 5% EtOH | — | — | 5 | TD | — | — |

Acetyl-cysteine/hypertonic pre-treatment followed by dosing in isotonic conditions at neutral pH
C57Bl/6 mice DOB Jun. 18, 2008 received progesterone treatment on Aug. 7, 2008 experiment begins Aug. 12, 2008 for 48 hr TD, Aug. 13, 2008 for 24 hr TD
mice were anesthetized and dosed with 30 ul 10 mg/ml acetyl-cysteine in 1XPBS
liquid/mucous is removed from vaginal canal and 30 ul of 2XPBS (548mOsmole) is adminstered
liquid/mucous is removed from vaginal canal and residual fluid removed with swab
10 ul formulation administered
Cytobrush inserted and twisted 10 times to disrupt epithelium
10 ul formulation administered
Take Down
10 mice per group: vaginal canal and cervix frozen separately for bDNA analysis

TABLE 11
Cholesterol Conjugated siRNAs Evaluated in Efficacy Experiments

| Duplex # | Target | Description | Strand | ssRNA # | Sequence 5′-3′ |
|---|---|---|---|---|---|
| 3175 | Keratin14 | 2′Fluoro modified Chol-siRNA | S | 30098 | GUfAUfUfGUfCfCfCfUfAUfAGGCfUfUfdTdTL10 |
| | | | AS | 22346 | AAGCfCfUfAUfAGGGACfAAUfACfdTsdT |
| 8885 | E6AP | 2′OMe modified Chol-siRNA | S | 13204 | AcGAAuGAGuuuuGuGcuudTdTL10 |
| | | | AS | 13161 | AAGcAcAAAACUcAuUCGUdTsdT |

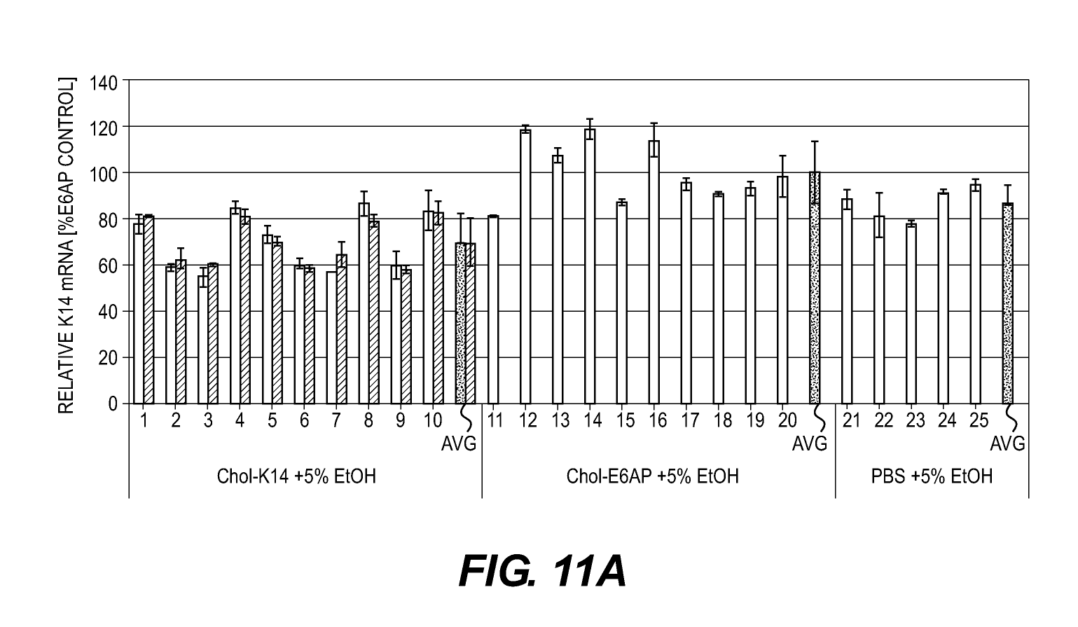

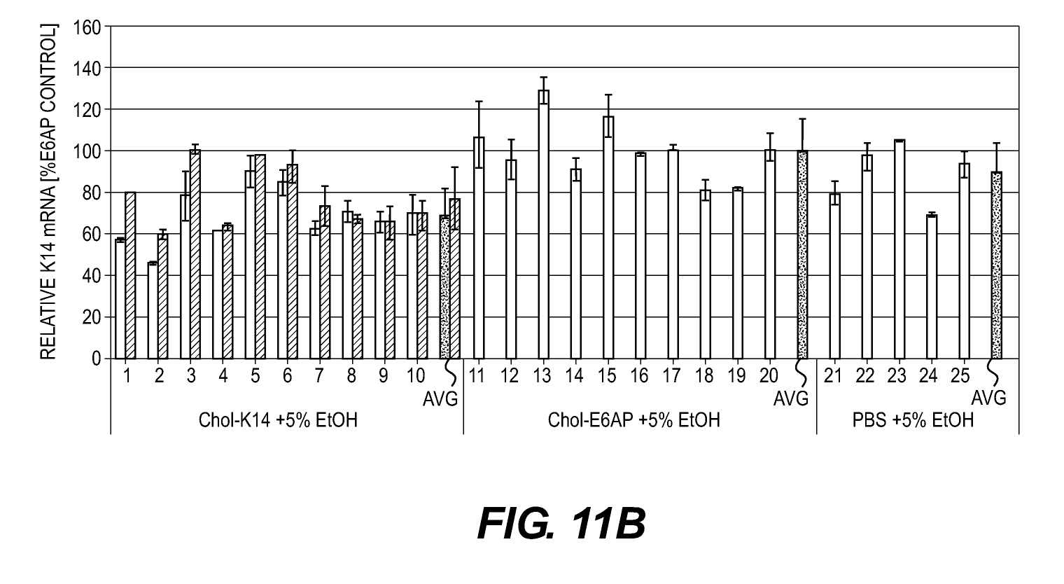

According to FIG. 11, slightly higher K14 KD was observed at 24 h vs. 48 h with K14-Chol siRNA in 5% ethanol. At 24 h, ˜31% K14 KD was observed with K14-Chol siRNA treatment relative to E6AP-chol siRNA treatment and ˜20% K14 KD was observed relative to PBS treatment (FIG. 11a). At 48 h, ˜32% K14 KD was observed with K14-Chol siRNA treatement relative to E6AP-chol siRNA treatment and ˜14% K14 KD was observed relative to PBS (FIG. 11b). Two measurements were taken on different plates for each animal in the K14-Chol siRNA treatment group confirming no plate effect.

### Example 7

### Evaluation of siRNA Efficacy In Vivo of Chol-K14 siRNA w/Cytobrush®, C57BL/6 vs Balb/C

TABLE 12
Cholesterol Conjugated siRNA + 5% EtOH Balb/C vs C57BL/6 mice

| | | | | | | Formulation | ug | |
|---|---|---|---|---|---|---|---|---|
| Group | Formulation | siRNA | Duplex # | N | Dosing | Concentration (siRNA) | siRNA/dose | Strain |
| 1 | K14-Chol + 5% EtOH | K14-Chol | 3175 | 10 | once/day 2 | 5 mg/ml | 100 | Balb/c |
| 2 | E6AP-Chol + 5% EtOH | E6AP-Chol | 8885 | 10 | days, 24 hr | | | |
| 3 | 1X PBS + 5% EtOH | — | — | 5 | TD | — | — | |
| 4 | K14-Chol + 5% EtOH | K14-Chol | 3175 | 10 | | 5 mg/ml | 100 | C57Bl/6 |
| 5 | E6AP-Chol + 5% EtOH | E6AP-Chol | 8885 | 10 | | | | |
| 6 | 1X PBS + 5% EtOH | — | — | 5 | | — | — | |
| | | | Total = | 50 | | | | |

Acetyl-cysteine/hypertonic pre-treatment followed by dosing in isotonic conditions at neutral pH
C57Bl/6 mice DOB Jun. 18, 2008 or Balb/C mice DOB Jun. 25, 2008 received progesterone treatment on Aug. 21, 2008, experiment begins Aug. 26, 2008
mice were anesthetized and dosed with 30 ul 10 mg/ml acetyl-cysteine in 1XPBS
liquid/mucous is removed from vaginal canal and 30 ul of 2XPBS (548mOsmole) is adminstered
liquid/mucous is removed from vaginal canal and residual fluid removed with swab
10 ul formulation administered
Cytobrush inserted and twisted 10 times to disrupt epithelium
10 ul formulation administered
Take Down
10 mice per group: vaginal canal and cervix frozen separately for bDNA analysis

TABLE 13
Cholesterol Conjugated siRNAs Evaluated in Efficacy Experiments

| Duplex # | Target | Description | Strand | ssRNA # | Sequence 5′-3′ |
|---|---|---|---|---|---|
| 3175 | Keratin14 | 2′Fluoro modified Chol-siRNA | S | 30098 | GUfAUfUfGUfCfCfCfUfAUfAGGCfUfUfdTdTL10 |
| | | | AS | 22346 | AAGCfCfUfAUfAGGGACfAAUfACfdTsdT |
| 8885 | E6AP | 2′OMe modified Chol-siRNA | S | 13204 | AcGAAuGAGuuuuGuGcuudTdTL10 |
| | | | AS | 13161 | AAGcAcAAAACUcAuUCGUdTsdT |

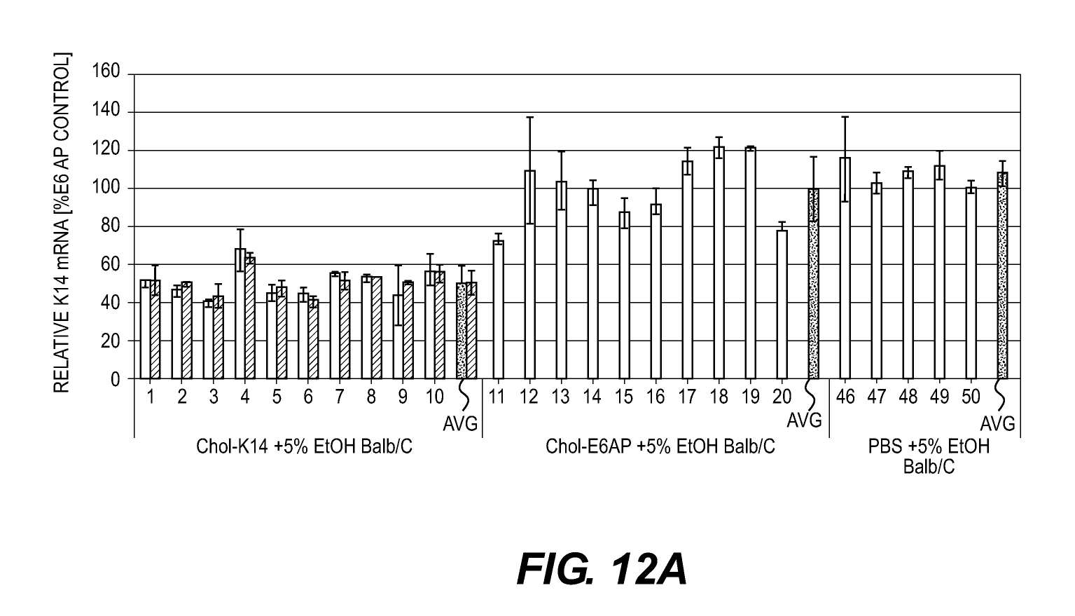

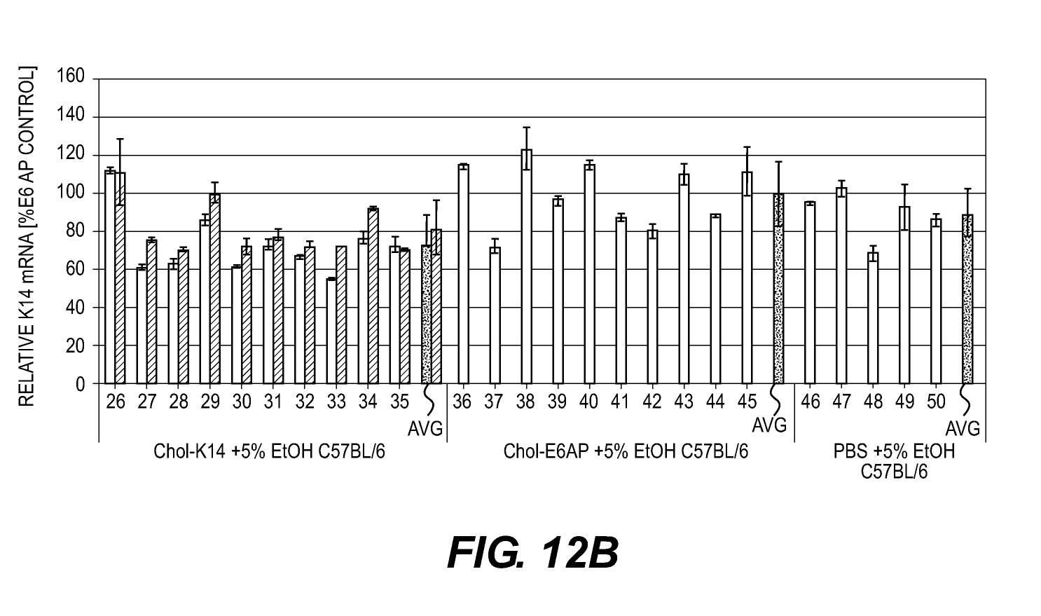

In a comparison study of two strains of mice, Balb/C and C57BL/6, it was determined that Balb/C mice show better in vivo efficacy than C57BL/6 mice. In the Balb/C mouse model, 50% K14 KD was observed with K14-Chol siRNA treatment relative to E6AP-Chol siRNA treatment or PBS treatment (FIG. 12a), while in the C57BL/6 mouse model, only 27% K14 KD was observed for K14-Chol siRNA treatment relative to E6AP-Chol siRNA treatment and only 19% K14 KD relative to PBS treatment (FIG. 12b). The higher observed KD of ˜30-45% KD, while not bound by theory, the inventors believe that the clear improvement in KD in Balb/C can be attributed to better cycling in Balb/cmice vs. C57BL/6 mice and in addition Balb/C mice might be more amenable to vaginal permeation as evidenced by this strains higher susceptibility to viral infection.

### Example 8

### Cytobrush® Requirement

+/− Cytobrush® with Chol-Conjugate+5% EtOH in Balb/C

TABLE 14

| | | | | | | | Formulation | ug |
|---|---|---|---|---|---|---|---|---|
| Group | Formulation | Cytobrush | siRNA | Duplex # | N | Dosing | Concentration (siRNA) | siRNA/dose |
| 1 | K14-Chol + 5% EtOH | Yes | K14-Chol | 3175 | 10 | once/day 2 | 5 mg/ml | 100 |
| 2 | LaminAC-Chol + 5% EtOH | | LaminAC-Chol | 3129 | 10 | days, 24 hr | | |
| 3 | 1X PBS + 5% EtOH | | — | — | 5 | TD | — | — |
| 4 | K14-Chol + 5% EtOH | No | K14-Chol | 3175 | 10 | | 5 mg/ml | 100 |
| 5 | LaminAC-Chol + 5% EtOH | | LaminAC-Chol | 3129 | 10 | | | |
| 6 | 1X PBS + 5% EtOH | | — | — | 5 | | — | — |
| | | | | Total = | 50 | | | |

Acetyl-cysteine/hypertonic pre-treatment followed by dosing in isotonic conditions at neutral pH
Balb/c mice DOB X/XX/08 received progesterone treatment on Sep. 11, 2008, experiment begins Sep. 17, 2008
mice were anesthetized and dosed with 30 ul 10 mg/ml acetyl-cysteine in 1XPBS
liquid/mucous is removed from vaginal canal and 30 ul of 2XPBS (548mOsmole) is adminstered
liquid/mucous is removed from vaginal canal and residual fluid removed with swab
For Cytobrush animals:
10 ul formulation administered
Cytobrush inserted and twisted 10 times to disrupt epithelium
10 ul formulation administered
For non-cytobrush animals:
20 ul formulation administered
Take Down
10 mice per group: vaginal canal and cervix frozen separately for bDNA analysis

TABLE 15

| Duplex # | Target | Description | Strand | sSRNA # | Sequence 5′-3′ |
|---|---|---|---|---|---|
| 3175 | Keratin14 | 2′Fluoro modified Chol-siRNA | S | 30098 | GUfAUfUfGUfCfCfCfUfAUfAGGCfUfUfdTdTL10 |
| | | | AS | 22346 | AAGCfCfUfAUfAGGGACfAAUfACfdTsdT |
| 3129 | LaminAC | Unmodified Chol-siRNA | S | 3894 | GAAGCAGCUUCAGGAUGAGdTsdTL10 |
| | | | AS | 3895 | CUCAUCCUGAAGCUGCUUCdTsdT |

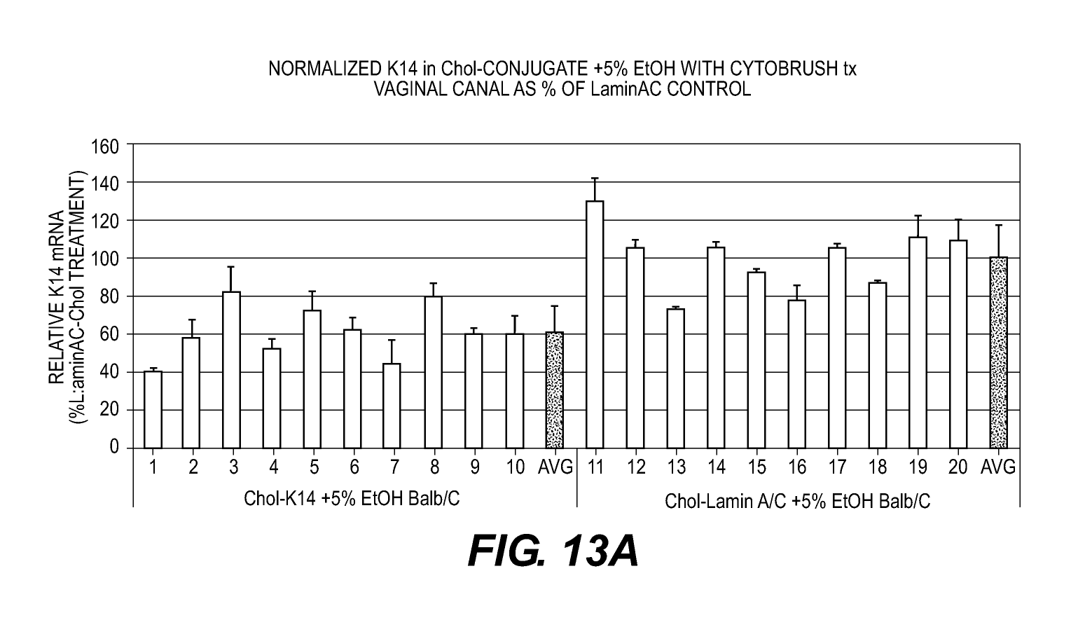

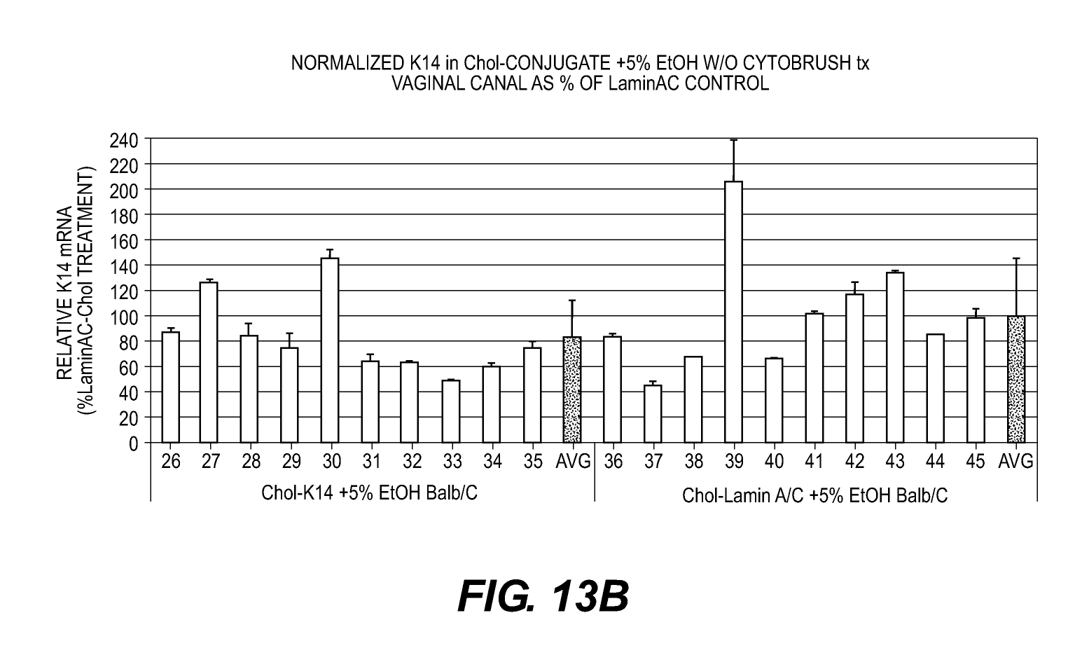

FIG. 13 illustrates the mechanical abrasion with regard to the observed knockdown. ˜40% K14 KD for K14-Chol siRNA treatment relative to LaminAC-Chol siRNA treatment was observed with aid of a Cytobrush® (FIG. 13a) and no significant K14 KD was observed for K14-Chol siRNA treatment relative to LaminAC-Chol siRNA treatment without a Cytobrush®. This experiment also illustrates specificity of silencing since no non-specific K14 KD is observed when animals are treated with a LaminAC-Chol siRNA.

### Example 9

### Specificity Study K14-Chol siRNA Relative to Nectin-Chol and E6AP-Chol siRNAs

TABLE 16

| | | | | | | Formulation | ug |
|---|---|---|---|---|---|---|---|
| Group | Formulation | siRNA | Duplex # | N | Dosing | Concentration (siRNA) | siRNA/dose |
| 1 | K14-Chol + 5% EtOH | K14-Chol | 3175 | 10 | once/day 2 | 5 mg/ml | 100 |
| 2 | E6AP-Chol + 5% EtOH | E6AP-Chol | 8885 | 10 | days, 24 hr | | |
| 3 | Nectin-Chol + 5% EtOH | Nectin-Chol | 3159 | 5 | TD | | |
| | | | Total = | 25 | | | |

Acetyl-cysteine/hypertonic pre-treatrnent followed by dosing in isotonic conditions at neutral pH
Balb/c mice DOB Jul. 30, 2008 received progesterone treatment on Sep. 18, 2008, experiment begins Sep. 24, 2008
mice were anesthetized and dosed with 30 ul 10 mg/ml acetyl-cysteine in 1XPBS
liquid/mucous is removed from vaginal canal and 30 ul of 2XPBS (548mOsmole) is adminstered
liquid/mucous is removed from vaginal canal and residual fluid removed with swab
10 ul formulation administered
Cytobrush inserted and twisted 10 times to disrupt epithelium
10 ul formulation administered
Take Down
vaginal canal and cervix frozen separately for bDNA analysis

TABLE 17

| Duplex # | Target | Description | Strand | ssRNA # | Sequence 5′-3′ |
|---|---|---|---|---|---|
| 3175 | Keratin14 | 2′Fluoro modified Chol-siRNA | S | 30098 | GUfAUfUfGUfCfCfCfUfAUfAGGCfUfUfdTdTL10 |
| | | | AS | 22346 | AAGCfCfUfAUfAGGGACfAAUfACfdTsdT |
| 8885 | E6AP | 2′OMe modified Chol-siRNA | S | 13204 | AcGAAuGAGuuuuGuGcuudTdTL10 |
| | | | AS | 13161 | AAGcAcAAAACUcAuUCGUdTsdT |
| 3159 | Nectin | Unmodified Chol-siRNA | S | 3968 | CCUGCAUUGUCAACUAUCAdTdTsL10 |
| | | | AS | 3967 | UGAUAGUUGACAAUGCAGGdTsdT |

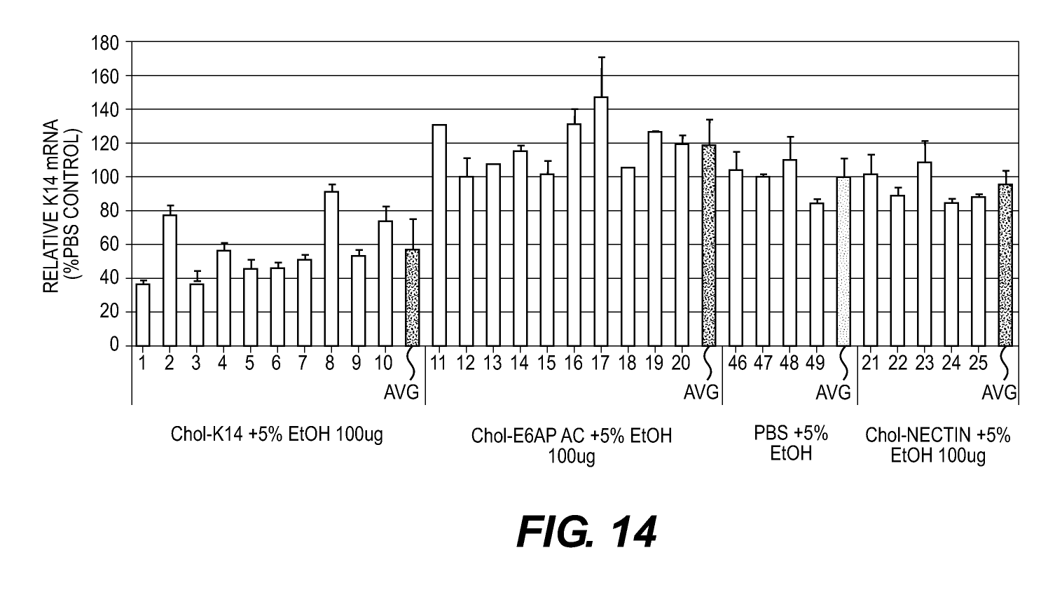

According to FIG. 14, no non-specific K14 KD was observed with either E6AP-Chol siRNA or Nectin-Chol siRNA treatment. ˜43% K14 KD was measured for K14-Chol siRNA treatment relative to the PBS control group.

### Example 10

### Free Uptake Conjugate Screen: HEK-A

### Protocol:

96-well format, Cells plated 24 hours prior to treatment

Conjugates diluted in Keratinocyte Growth Media (Serum-Free), and layered on top of cells

Free Uptake=9 uM, 1.8 uM, and 0.36 uM vs. 9 uM 1956 Luc-Chol

24 hour-incubation, no media renewal

bDNA readout for efficacy

TABLE 18

| duplex | | |
|---|---|---|
| Name | Sense strand (5′-3′) | antisense strand (5′-3′) |
| 8816 | AcGAAuGAGuuuuGuGcuudTsdT | AAGcAcAAAACUcAuUCGUdTsdT |
| 8885 | AcGAAuGAGuuuuGuGcuudTdTL10 | AAGcAcAAAACUcAuUCGUdTsdT |
| 19155 | AcGAAuGAGuuuuGuGcuudTdTL98 | AAGcAcAAAACUcAuUCGUdTsdT |
| 19157 | AcGAAuGAGuuuuGuGcuudTdTL92 | AAGcAcAAAACUcAuUCGUdTsdT |
| 21163 | AcGAAuGAGuuuuGuGcuudTdTL55 | AAGcAcAAAACUcAuUCGUdTsdT |
| 21164 | AcGAAuGAGuuuuGuGcuudTdTL60 | AAGcAcAAAACUcAuUCGUdTsdT |
| 21165 | AcGAAuGAGuuuuGuGcuudTdTL57 | AAGcAcAAAACUcAuUCGUdTsdT |
| 21166 | AcGAAuGAGuuuuGuGcuudTdTL54 | AAGcAcAAAACUcAuUCGUdTsdT |
| 21167 | AcGAAuGAGuuuuGuGcuudTdTL13 | AAGcAcAAAACUcAuUCGUdTsdT |
| 21168 | AcGAAuGAGuuuuGuGcuudTdTL116 | AAGcAcAAAACUcAuUCGUdTsdT |
| 21169 | AcGAAuGAGuuuuGuGcuudTdTL58 | AAGcAcAAAACUcAuUCGUdTsdT |
| 21170 | AcGAAuGAGuuuuGuGcuudTdTL122 | AAGcAcAAAACUcAuUCGUdTsdT |
| 21164 | AcGAAuGAGuuuuGuGcuudTdTL60 | AAGcAcAAAACUcAuUCGTUdTsd |
| 22584 | AcGAAuGAGuuuuGuGcuudTdTL144 | AAGcAcAAAACUcAuUCGUdTsdT |

Linoelyl E6AP (L55)
Oleyl E6AP (L60)
Stearyl E6AP (L57)
Palmityl E6AP (L54)
Vitamin E E6AP (L13)
Lithocholic Acid E6AP (L116)
Docasonyl E6AP (L58)
Cholesteroylamine (L122)
S-S Oleyl (L144)

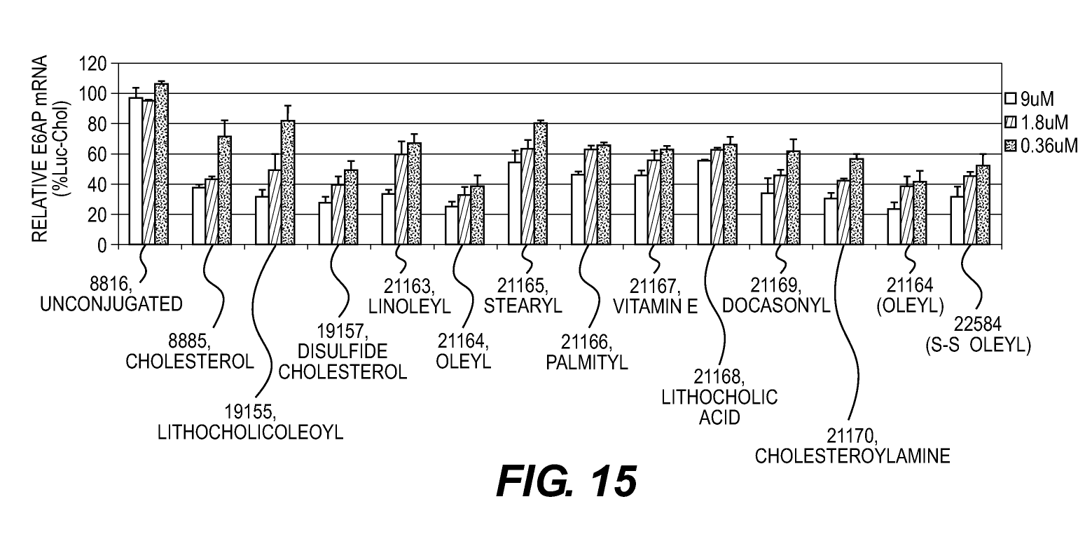

According to FIG. 15, siRNA with various conjugates show efficacy in human primary keratinocytes relative to the unconjugated siRNA, with good results seen in many of the conjugates, in particular, the oleyl conjugates.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

We claim:

1. A method for enhancing delivery of an oligonucleotide into and across one or more layers of an animal epithelial tissue, the method comprising administering to the epithelial tissue one or more lipophilic conjugates selected from the group consisting of disulfide-steroid, PEG-steroid, aliphatic chain, phospholipid, polyamine chain, polyethylene glycol chain, and combinations thereof.

2. The method of claim 1, wherein the lipophilic conjugate is oleyl, disulfide-oleyl, disulfide-cholesterol, C22, C 16-cholesterol, lithocholicoleoyl, or PEG4-cholesterol.

3. The method of claim 1, further comprising a penetration enhancer.

4. The method of claim 3, wherein the penetration enhancer is selected from the group consisting of alcohols, surfactants, fatty acids, polyols, amides, and sulfoxides.

5. The method of claim 3, wherein the penetration enhancer is ethanol.

6. The method of claim 1, further comprising a mechanical enhancer.

7. The method of claim 3, further comprising a mechanical enhancer.

8. The method of claim 1, wherein the epithelial tissue is in the vaginal cannal.

9. The method of claim 1, wherein the delivery is selected from topical, electroporation, intradermal or epidermal injection.

10. A composition for use in delivering an oligonucleotide across the epithelial tissue, comprising a lipophilic-conjugated oligonucleotide, wherein the lipophilic conjugate of the lipophilic-conjugated oligonucleotide is selected from the group consisting of disulfide-steroid, PEG-steroid, aliphatic chain, phospholipid, polyamine chain, and polyethylene glycol chain.

11. The composition of claim 10, wherein the oligonucleotide contains oleyl, disulfide-oleyl, disulfide-cholesterol, C22, C16-cholesterol, lithocholicoleoyl, a PEG4-cholesterol, or a combination thereof.

12. The composition of claim 10, further comprising a penetration enhancer.

13. The composition of claim 12, wherein the penetration enhancer is selected from the group consisting of alcohols, surfactants, fatty acids, polyols, amides and sulfoxides.

14. The composition of claim 12, wherein the penetration enhancer is ethanol.

15. The composition of claim 10, further comprising a mechanical enhancer.

16. The composition of claim 12, further comprising a mechanical enhancer.

17. The composition of claim 10, wherein the epithelial tissue is in the vaginal canal.

18. A composition for delivery of oligonucleotides across and into the epithelial tissues, comprising a lipophilic-conjugated oligonucleotide, a penetration enhancer, and a mechanical enhancer.

19. The composition of claim 18, wherein the lipophilic conjugate of the lipophilic-conjugated oligonucleotide is selected from the group consisting of oleyl, disulfide-oleyl, cholesterol, disulfide-cholesterol, C22, lithocholicoleoyl, and PEG4-cholesterol.

20. The composition of claim 18, wherein the penetration enhancer is ethanol.

21. A composition comprising one or more lipophilic-conjugated oligonucleotides, wherein the oligonucleotides have been formulated for electroporation into cells in vivo.

22. The composition of claim 21, wherein the lipophilic conjugate of the lipophilic-conjugated oligonucleotide is selected from the group consisting of oleyl, disulfide-oleyl, cholesterol, disulfide-cholesterol, C22, lithocholicoleoyl, and PEG4-cholesterol.

23. The composition of claim 21, wherein the lipophilic conjugate of the lipophilic-conjugated oligonucleotide is formulated in supramolecular complexes or liposomes.

24. The composition of claim 21, wherein the cells are epithelial cells.

25. A method for delivering one or more lipophilic conjugates to a patient by intradermal injection, transdermal injection, or epidermal injection to the epithelial tissues, comprising administering a sufficient amount of the lipophilic conjugate to an animal, wherein the lipophilic conjugate attenuates expression of a target gene in cells of the animal.

26. The method of claim 25, wherein the lipophilic conjugate is selected from the group consisting of cholesterol, oleyl, disulfide-oleyl, disulfide-cholesterol, C22, lithocholicoleoyl, and PEG4-cholesterol.
